(19) United States
(12) Patent Application Publication
(10) Pub. No.: US 2024/0245886 A1
(43) Pub. Date: Jul. 25, 2024

(54) MULTI-DEGREE-OF-FREEDOM STEERABLE CATHETER SOFT ROBOTIC SYSTEM, METHODS OF MANUFACTURING A STEERABLE CATHETER, AND OPERATING THE SAME

(71) Applicant: National Tsing Hua University, Hsinchu City (TW)

(72) Inventors: Wei-Chih Wang, Sammamish, WA (US); Fiona Marie Wang, Seattle, WA (US); Hsiao-Ya Chu, Hsinchu City (TW); Yu Yang Hsu, Hsinchu City (TW); Chun-Yen Hsieh, Hsinchu City (TW)

(21) Appl. No.: 18/159,591

(22) Filed: Jan. 25, 2023

(51) Int. Cl. A61M 25/01; A61B 34/30

(52) U.S. Cl. CPC A61M 25/0105; A61B 34/30; A61B 2034/301

(57) ABSTRACT

Provided is a multi-degree-of-freedom steerable catheter soft robotic system, including a steerable catheter; a control circuit connected to the steerable catheter through electrical connections and selectively applying power to control the steerable catheter; and a power supply unit connected to the control circuit. The system also includes a driving circuit for driving the steerable catheter and a shielding disposed around the steerable catheter and shielding for heat and electromagnetic (EM) radiations. The present disclosure includes self-sensing shape-shifting spring coil actuators and a shape-shifting memory polymer (SMP) actuator for steerable catheter applications. In addition, the present disclosure also provides an electroless silver plating process, a silver chemical plating process, a carbon nanotube (CNT) composite process and a pneumatic process.

### BACKGROUND

### 1. Technical Field

The present disclosure relates to catheter designs and, more specifically, to a multi-degree-of-freedom steerable catheter soft robotic system, methods of manufacturing a steerable catheter, and operating the same.

### 2. Description of Related Art

Nowadays, shape memory alloy (SMA)-based catheters have been used in the medical environment to provide intravenous vascular access. Vascular access by the catheters provides a convenient and highly effective means for various applications, such as drug administration, fluid administration, chemotherapy, blood sampling, blood pressure monitoring and parenteral nutrition.

The above-mentioned applications often require that the catheter would be left indwelling in a patient for a period of time. In the medical applications, catheters are also widely used in emergency departments, intensive care units or operating rooms. In such applications, these catheters must be correctly and quickly positioned intravenously within a patient to obtain vascular access, particularly in some critical situations concerning rapid drug administration and surgery.

However, the current SMA-based catheters cannot achieve a semi-autonomous navigation through complex vascular shapes.

As such, improved catheter systems and methods of fabricating a steerable catheter for a semi-autonomous navigation are needed.

### SUMMARY

The present disclosure provides a multi-degree-of-freedom steerable catheter soft robotic system, which includes a steerable catheter; a control circuit connected to the steerable catheter through electrical connections and selectively applying power to control the steerable catheter; and a power supply unit connected to the control circuit.

In an embodiment of the present disclosure, the system further includes a driving circuit for driving the steerable catheter and a shielding disposed around the steerable catheter and shielding for heat and electromagnetic (EM) radiations.

In an embodiment of the present disclosure, the steerable catheter includes a plurality of tubular segments, and each of the tubular segments includes a plurality of self-sensing shape-shifting spring coil actuators. The plurality of self-sensing shape-shifting spring coil actuators are evenly spaced inside each of the tubular segments.

In an embodiment of the present disclosure, a seed layer is deposited on a surface of each of the plurality of self-sensing shape-shifting spring coil actuators by an electroless silver plating process, and a silver layer is deposited on a surface of the seed layer by the electroless silver plating process.

In another embodiment of the present disclosure, the steerable catheter includes a shape-shifting memory polymer (SMP) actuator. The SMP actuator may be coated with conductive coating agents by a silver chemical plating process such that the SMP actuator is a self-sensing silver plated SMP actuator. Alternatively, the SMP actuator does not need silver on top since it is already conductive by itself. Adding silver helps improve the overall performance by lowering the current to drive the actuator and easier to wire the actuator. In addition, the SMP actuator may be made electrically and thermally conductive by a carbon nanotube (CNT) composite process such that the SMP actuator is a self-sensing CNT-based SMP actuator.

In another embodiment of the present disclosure, the SMP actuator is bent upward or downward to show flexibility by utilizing a multi-phase shape-shifting memory material, and the multi-phase shape-shifting memory material may be a self-sensing and reversible LC elastomer. In addition, the multi-phase shape-shifting memory material may be a self-sensing and reversible bi-layer composite sheet, and the self-sensing and reversible bi-layer composite sheet comprises a self-sensing and reversible CNT-based SMP together with a polyurethane (PU), polyimide (PI) or polyester (PET) film.

In another embodiment of the present disclosure, a Negative Poisson's Ratio (NPR) structure is used in the SMP actuator such that auxetics and strains of the SMP actuator are enhanced.

Moreover, the present disclosure also provides a method of manufacturing a steerable catheter of a multi-degree-of-freedom steerable catheter soft robotic system. The method includes: forming a plurality of tubular segments; forming a plurality of self-sensing shape-shifting spring coil actuators inside each of the plurality of tubular segments; depositing a seed layer on a surface of each of the plurality of self-sensing shape-shifting spring coil actuators by an electroless silver plating process; and depositing a silver layer on a surface of the seed layer by the electroless silver plating process.

In an embodiment of the present disclosure, the electroless silver plating process includes: immersing a spring coil into sodium hydroxide (NaOH) in a beaker; placing the spring coil into an ultrasonic washing machine for few minutes; performing an Iodine pretreatment on the spring coil; immersing the spring coil an Au etchant under room temperature; placing the spring coil into sodium borohydride (NaBH4); and rinsing the spring coil to remove any chemicals and residue out of a surface thereof such that the seed layer is formed on a surface of the spring coil.

In an embodiment of the present disclosure, the electroless silver plating process further includes forming the silver layer on the surface of the seed layer such that the spring coil becomes the self-sensing shape-shifting spring coil actuator.

Additionally, the present disclosure further provides a method of manufacturing a steerable catheter of a multi-degree-of-freedom steerable catheter soft robotic system. The system includes forming and coating a self-sensing shape-shifting memory polymer (SMP) actuator without conductive coating agents or with conductive coating agents by a silver chemical plating process or a carbon nanotube (CNT) composite process.

In another embodiment of the present disclosure, the silver chemical plating process includes: providing a solution; pouring the solution into a mold; heating the mold by a curing process; forming a sheet after the solution is fully cured; removing the sheet from the mold; and cutting a window array pattern on the sheet, wherein the solution is one of a polyurethane (PU)-based shape memory polymer solution, a polyimide (PI)-based shape memory polymer solution and a polyester (PET)-based shape memory polymer solution.

In another embodiment of the present disclosure, the silver chemical plating process further includes: depositing a conductive layer on a surface of the sheet; and rolling the sheet into a tube.

In another embodiment of the present disclosure, the window array pattern is a rectangular window array pattern, a re-entrant honeycomb window array pattern, a chiral honeycomb window array pattern, a rotating rectangle window array pattern or a combination thereof.

In an alternative embodiment of the present disclosure, the CNT composite process includes: providing a solution; pouring the solution into a mold; heating the mold by a curing process; forming a sheet after the solution is fully cured; removing the sheet from the mold; and cutting a window array pattern on the sheet, wherein the solution is provided by mixing liquid phase SMP, dimethylformamide (DMF) and CNT powders together with a weight ratio.

In an alternative embodiment of the present disclosure, the CNT composite process further includes: depositing a conductive layer on a surface of the sheet; and rolling the sheet into a tube.

In the present disclosure, the sheet is one of a self-sensing and reversible LC elastomer and a self-sensing and reversible bi-layer composite sheet.

In the present disclosure, the self-sensing and reversible bi-layer composite sheet comprises a self-sensing and reversible CNT-based SMP together with a polyurethane (PU), polyimide (PI) or polyester (PET) film.

Besides, the present disclosure provides a method of operating a steerable catheter of a multi-degree-of-freedom steerable catheter soft robotic system. The method includes providing a silicone tube and a steerable catheter: connecting a portion of the silicon tube to a nitrogen gas tank with a pressure control valve and connecting another portion of the silicone tube to into steerable catheter; releasing nitrogen gas into the silicon tube to enhance stiffness of the silicon tube; and gradually increasing an air pressure to straighten the steerable catheter.

In the method of the present disclosure, when no gas is released into the silicon tube, the silicon tube remains soft such that the steerable catheter is bent upward or downward, and the steerable catheter is a self-sensing and reversible carbon nanotube (CNT)-based SMP actuator.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a steerable catheter system.

FIG. 1B shows five 2 cm long 2.33 mm diameter tubular links.

FIG. 1C is an alternated PU polymer all-tubular catheter.

FIG. 1D shows a 4 shape-shifting spring actuator array.

FIG. 2A shows a 2.5 mm diameter four stage cylindrical actuator in various actuated bending positions.

FIG. 2B shows spring contraction as a function of temperature (500 μm diameter spring, original length 27 mm, stretched to 37 mm by 25 g weight).

FIG. 2C shows a time transient of a spring's contraction and pull force as a function of temperature of the 500 μm diameter spring.

FIG. 2D shows a displacement-inductance plot, wherein the blue line represents when displacement is increasing and the orange line represents when displacement is decreasing. From the result, inductance is positively proportional to displacement.

FIG. 2E shows capacitance as a function of temperature.

FIG. 2F shows a displacement-resistance diagram.

FIG. 3A is an optical image showing a precursor fiber.

FIG. 3B is an optical image showing chiral fiber after twisted.

FIG. 3C is an optical image showing an artificial muscle with a hierarchical chiral structure after a twisting-then-coiling procedure.

FIG. 4 is a semi-crystalline structure of the polyethylene and nylon.

FIG. 5A is a push-pull mechanism actuator.

FIG. 5B shows that the whole actuator is bent to the left when the left spring is activated.

FIG. 5C shows that the actuator consists of four springs.

FIG. 5D shows a three-section actuator system displaying a snake-like movement.

FIG. 5E shows a multiple spring snake actuator controlled by the joystick and driving circuit.

FIG. 5F shows 2-stage actuation: (a) bottom stage bending to the left, (b) no bending, (c) bottom stage starting to bend to the right, (d) bottom stage bending even more to the right, and (e) both top and bottom stages bending to the right.

FIG. 6A shows a twisted-then-coiled spring fabrication setup.

FIG. 6B shows an enlarged view of the twisted-then-coiled spring fabrication setup.

FIG. 7 is a nylon spring that fully absorbs iodine ions.

FIG. 8 is the nylon spring with a thin silver seed layer after pretreatment.

FIG. 9 is a conductive nylon spring after an electroless silver coating process.

FIG. 10 shows enlarged views of a silver-plated nylon spring (SEM photography).

FIG. 11A shows a demonstration of a 6 cm long and 4 mm in diameter PU based heat activated tubular SMP actuator bending 270°.

FIG. 11B shows the same PU tubular actuator with different pre-shape memory bending 82° and contracting 20% in length simultaneously during actuation.

FIG. 11C shows a 3D printed model of femoral arteries, abdominal aorta and splenic, proper and common hepatic arteries.

FIG. 11D shows a catheter evaluation test environment using a Amigo robot.

FIG. 12A shows a curing process of a solution type SMP.

FIG. 12B shows a SMP sheet with 400 μm after curing.

FIG. 13 shows geometry of the SMP sheet with a rectangular window array pattern.

FIG. 14A shows a straight configuration of a rectangular window patterned tube.

FIG. 14B shows a bent configuration of the rectangular window patterned tube.

FIG. 15A shows that a tubular shape memory polymer actuator with a permanent shape above 120°.

FIG. 15B shows the tubular shape memory polymer actuator deformed by an external force and staying in a temporary shape under room temperature.

FIG. 15C shows the shape recovery of the tubular shape memory polymer actuator once it is re-related above the glass transition temperature.

FIG. 16 shows a CNT-based SMP solution shear casted in an aluminum mold.

FIG. 17 shows a CNT-based SMP tube with a rectangular window pattern.

FIG. 18A shows that the CNT/SMP composite is poured into the PDMS mold and bakes for 2 hours at 100° C.

FIG. 18B shows that a resulting film has a resistance value in the 10′sk Ω range once it is cured and removed.

FIG. 19A shows a stretching load test when the displacement is 0 mm.

FIG. 19B shows a stretching load test when the displacement is 10 mm.

FIG. 20 is a displacement-force schematic diagram of a CNT-based SMP tube with a larger window and a smaller gap pattern.

FIG. 21 is a displacement-resistance schematic diagram of a CNT-based SMP tube with a larger window and a smaller gap pattern.

FIG. 22 shows a pure SMP patterned tube with a thin layer of silver using sputter.

FIG. 23 shows a silver epoxy-coated patterned SMP sheet before rolling up into a tube configuration.

FIG. 24 shows a heat distribution of the silver sputtered tube under 10V using a 375 W power supply.

FIG. 25 shows a heat distribution of the silver epoxy-coated SMP patterned tube under 4.8 V, 0.15 A.

FIG. 26 shows a heat distribution of the CNT-based SMP patterned tube under 42 V, 0.025 A.

FIG. 27 is a time versus current and temperature diagram of the CNT-based SMP tube under 20 V.

FIG. 28 is a time versus current and temperature diagram of the CNT-based SMP tube under 30 V.

FIG. 29 is a time versus current and temperature diagram of the CNT-based SMP tube under 40 V.

FIG. 30 is a time versus current and temperature diagram of the CNT-based SMP tube under 45V.

FIG. 31A is a stacked picture showing twisting actuation after applying 30 V for 30 seconds.

FIG. 31B shows a temperature distribution on the CNT-based SMP tube after applying 30 V for 30 seconds during twisting actuation.

FIG. 32A is a stacked picture showing twisting actuation from the beginning to applying 45 V on the CNT-based SMP tube.

FIG. 32B shows a temperature distribution on the CNT-based SMP tube after applying 45 V.

FIG. 33A is a CNT-based SMP tube with three electrodes in a straight permanent shape.

FIG. 33B shows a retracted area and a bent area of the CNT-based SMP tube.

FIG. 34A shows a partially control upper part of the CNT-based SMP tube using 13.9 V.

FIG. 34B shows a temperature configuration of the whole tube when only applied 13.9 V to the upper section of the tube.

FIG. 35A shows a partially control lower part of the CNT-based SMP tube using 13.9 V.

FIG. 35B shows a temperature configuration of the whole tube when only applied 13.9 V to the lower section of the tube.

FIG. 36A shows a partially control upper part of the CNT-based SMP tube using 20 V.

FIG. 36B shows a temperature distribution of the whole tube when only applied 20 V to the upper section of the tube.

FIG. 37 shows that a bi-layer sheet include CNT-based SMP and a PI or PET film.

FIG. 38A shows an original straight temporary shape of the CNT-based SMP tube.

FIG. 38B shows the CNT-based SMP tube started to bend after heated upon 58.5° C.

FIG. 38C shows the CNT-based SMP tube bent downward when voltage is set to 0 and started to add air pressure.

FIG. 39 shows that the bi-layer cantilever device achieves a shape recovery of 50° by applying 20V.

FIG. 40 shows a two-way shape memory behavior in the CNT-SMP beam-type actuator.

FIG. 41 shows an example of a bending or multi-DOF actuator made of two of these bi-layer cantilever strips where the two-way actuation can be achieved with on or multistage actuator design.

FIG. 42 shows that the bending or multi-DOF actuator is achieved by implementing these bi-layer CNT-SMP/PI strips to generate large two-way (i.e., reversible) bending or extension motion.

FIG. 43 shows a 3D model of the SMP linkage design.

FIG. 44 shows a SMP simulation (initial state).

FIG. 45 shows bending of 5 segments of the linkage design.

FIG. 46 shows an initial state of the reversible SMP linkage design.

FIG. 47A shows a maximum upper bending angle of the reversible SMP linkage design.

FIG. 47B shows a maximum lower bending angle of the reversible SMP linkage design.

FIG. 48 shows that the PI film and CNT-based SMP is put into a mold, and shear casted with a glass slide.

FIG. 49 shows the film is cut into the same size, and silver glue is used to fix both ends of the test piece to fix the copper wire.

FIG. 50 shows that the film is fixed on a metal cylinder, and heated in an oven at 140 degrees for 5 minutes.

FIG. 51 shows that a curved permanent shape is formed.

FIG. 52 shows a dimension of a holder.

FIG. 53 shows a testing setup for the 2-way bi-layer CNT/SMP PI tubular actuator design.

FIG. 54A shows the reversible bi-layer CNT/SMP PI tubular actuator testing before heating.

FIG. 54B shows the reversible bi-layer CNT/SMP PI tubular actuator testing after heating.

FIG. 55A shows a 2D re-entrant honeycomb structure.

FIG. 55B shows a 2D chiral honeycomb structure.

FIG. 55C shows a 2D rotating rectangles structure.

FIG. 56A shows actuators based on a left handed rotating rectangles structure and a right handed rotating rectangles structure.

FIG. 56B shows an actuator with a NPR structure.

FIG. 57 shows a sleeve with a NPR structure.

FIG. 58 shows basic parameters in 2D re-entrant honeycomb geometry.

FIG. 59A shows the 2D re-entrant honeycomb structure at different degrees of strain in the y direction.

FIG. 59B shows that Poisson's ratio changes with the degrees of strain in the y direction.

FIG. 60 shows a 2D re-entrant honeycomb structure designed with SolidWorks for cutting.

FIG. 61A shows a Stencil cutting machine (SDX 125, Brother) used for fabricating.

FIG. 61B shows that a thickness of a HAP sheet is 600 μm attached to the mat before cutting.

FIG. 62A is a front view of the HAP sleeve.

FIG. 62B shows that the HAP sleeve is made by rolling up the 2D re-entrant honeycomb sheet, and fixed by melt.

FIG. 63 shows a dimension of the HAP sleeve (i.e., 120 mm long and 25 mm diameter).

FIGS. 64A and 64B show a demonstration of the NPR behavior under uniaxial tension.

FIGS. 65A and 65B show a tubular NPR actuator model to the sleeve device based on a 2D re-entrant honeycomb structure designed with SolidWorks.

FIG. 66A shows a tubular NPR structure printed along an axial direction.

FIG. 66B shows that a 3D printer is used (da Vinci 1.0 pro, XYZ printing).

FIG. 67A shows a tubular NPR structure printed along a transverse direction directly.

FIG. 67B shows a bottom view of the tubular NPR structure.

FIG. 68 shows that the tubular NPR actuator is printed on the base.

FIG. 69A shows a demonstration of the tubular NPR actuator.

FIG. 69B shows the tubular NPR actuator after an electroless plating process.

FIG. 70A shows that the tubular NPR actuator is pre-stressed before heating.

FIG. 70B show that the tubular NPR actuator tens to shift back to its initial shape after activated.

FIG. 71 shows a uniform rectangular patch.

FIGS. 72A-72C show cutting holes into a rectangular area.

FIGS. 73A-73C show a 4-cell structure.

FIGS. 74A-74C show a 64-cell structure.

FIG. 75A is a diagram showing maximum strain versus number of cells.

FIG. 75B is a diagram showing Poisson's ratio varying with number of cells.

FIG. 76 shows a CNT/SMP composite using strain enhance design.

FIG. 77A shows electrode masks.

FIG. 77B shows a CO₂ laser cut mask.

FIG. 78 shows a setup for depositing Au on a silicone tube.

FIG. 79A shows electrodes on a silicone tube.

FIG. 79B shows an enlarged view of the electrodes on the silicone tube.

FIG. 80A shows a spring load curve at 25° C.

FIG. 80B is a diagram showing a spring contraction as a function of temperature.

FIG. 80C shows a pull force versus temperature diagram of a spring actuator.

FIG. 81 shows a capacitance versus temperature diagram of a spring actuator.

### DETAILED DESCRIPTION

The embodiments of the present disclosure are related to the drawings for description hereinafter.

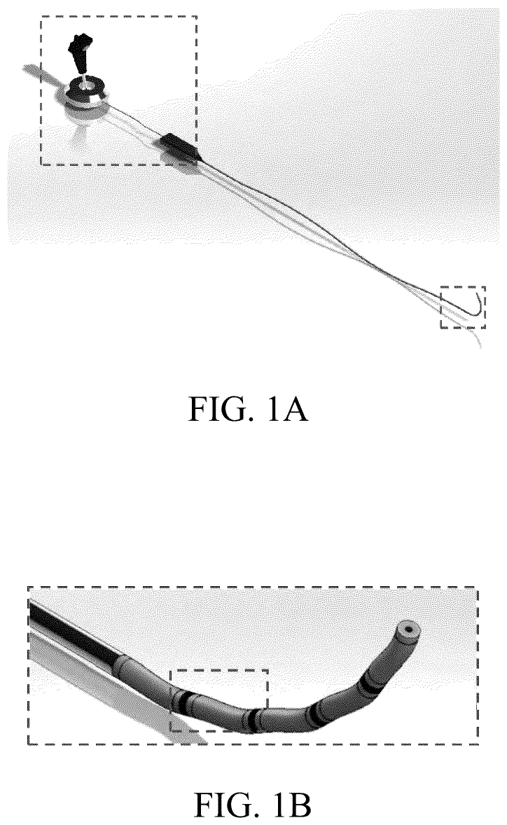

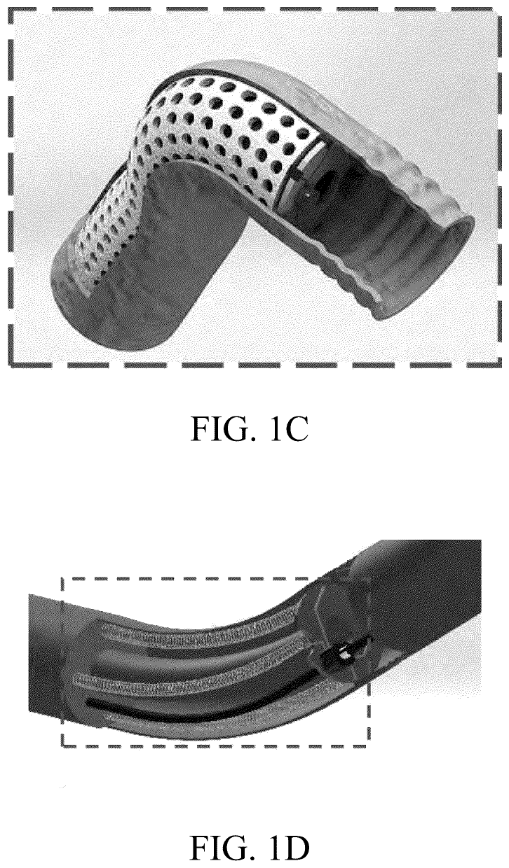

A smart active catheter is illustrated in FIG. 1A. It has a jointed chain structure with distributed actuators to achieve multiple independent degrees of freedom. The shape-shifting coils can activate independently, thereby allowing the tip region to ‘snake’ back and forth without guide wires binding. Each link has embedded integrated interface circuits with signal multiplexing and sensor and actuator control units such that the number of wires required for control can be minimized.

### First Embodiment

The operating principle is provided as follows. The catheter includes an elongated tubular member having a proximal end and a distal end for insertion into a targeted area (as shown in FIG. 1A). In the first embodiment of the present disclosure, the multilink-jointed structure consists of five segments with four evenly spaced polyimide (PI)-based shape-shifting spring coil actuators around each link. The shape-shifting materials behave like a shape memory polymer: returning to their original spring shape when heated above transition temperature, and deforming to a loaded form when cooled. The coil elements activate via current-induced heating, with each shape memory element coupled to at least one other element for extension-retraction movement (as shown in FIG. 1B). Each memory element is moved to a second shape when the series-coupled element is heated to the predetermined temperature by adjusting the actuation current. Each actuator is also a capacitive sensor such that a closed-loop feedback system to precisely control the tip movement can be created. A control system connected to the catheter allows an operator to selectively control the temperature of each element, thereby precisely directing the bending angle and direction of the catheter tip. The control system includes a power supply (e.g., lithium battery), electrical connections between the supper and the actuation/sensing elements, and the control circuit for selectively applying power to heat the actuators.

### Spring Actuator Design

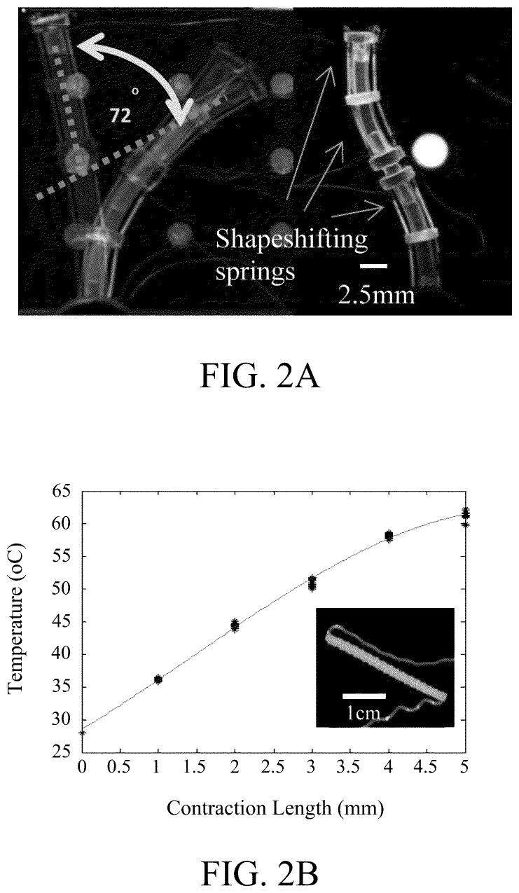

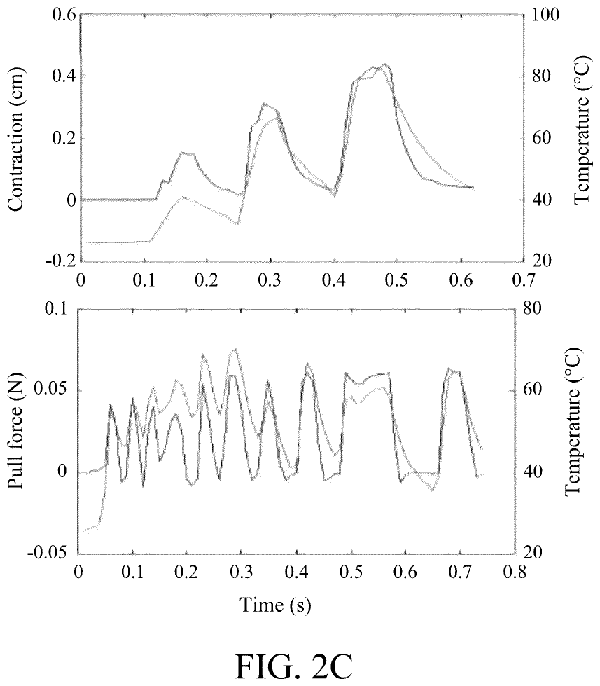

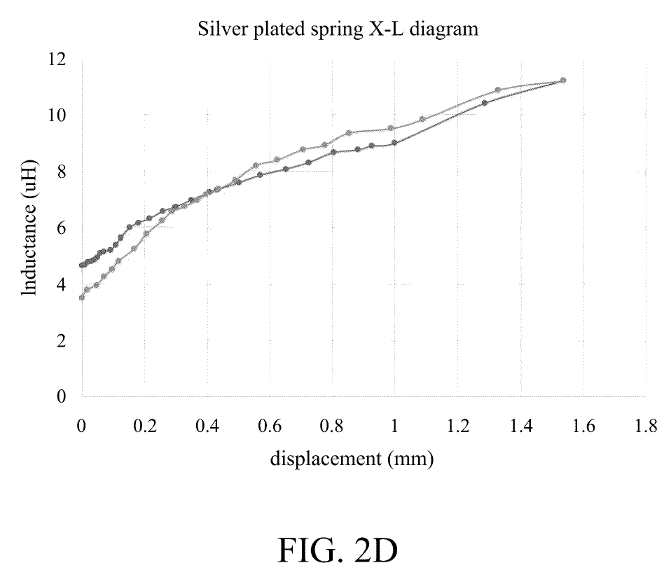

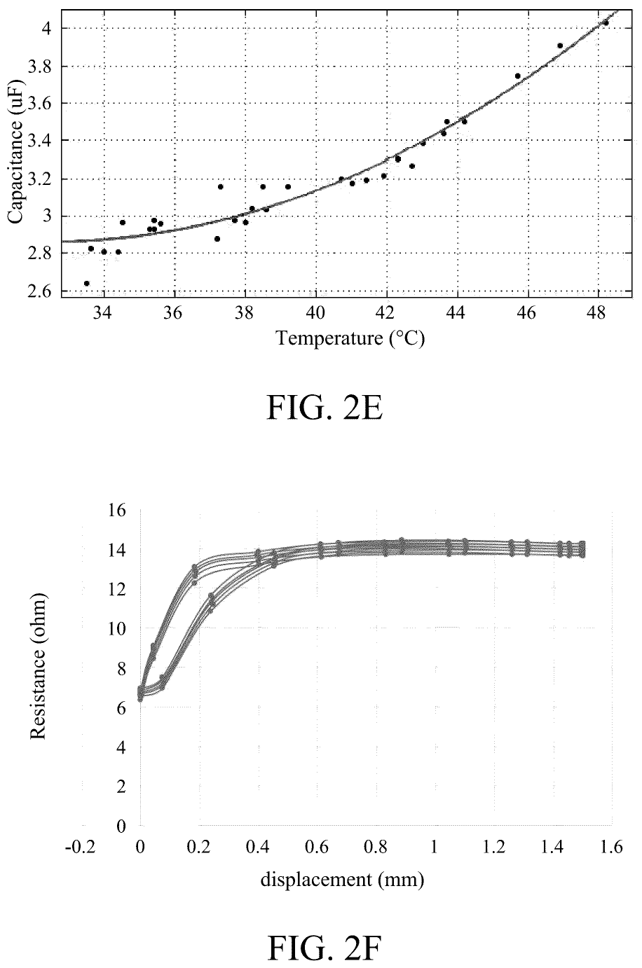

The actuator includes a polyamide-based shape-shifting polymer with shape memory characteristics. Shape memory materials undergo deformation when cooled below a specific temperature, and then recover their undeformed shape upon being heated above a transformation temperature. The actuator is a coil spring (as shown in FIG. 1B). The actuator is modified from an existing actuator that PI is previously developed for a soft robotic catheter system (as shown in FIGS. 1A and 2A). The actuator design (i.e., a 500 micro diameter spring formed from a 150 micro diameter wire) delivers very promising results of 37% spring contraction, with 0.5N axial support force, as shown in FIG. 2B. Additionally, it is demonstrated that consistent retraction behavior and pulling force are a function of temperature with low hysteresis (as shown in FIG. 2C), and a 79° bending angle is provided when the two of the four stages are activated with a 24 mW input power consumption.

### Spring Actuation Principle

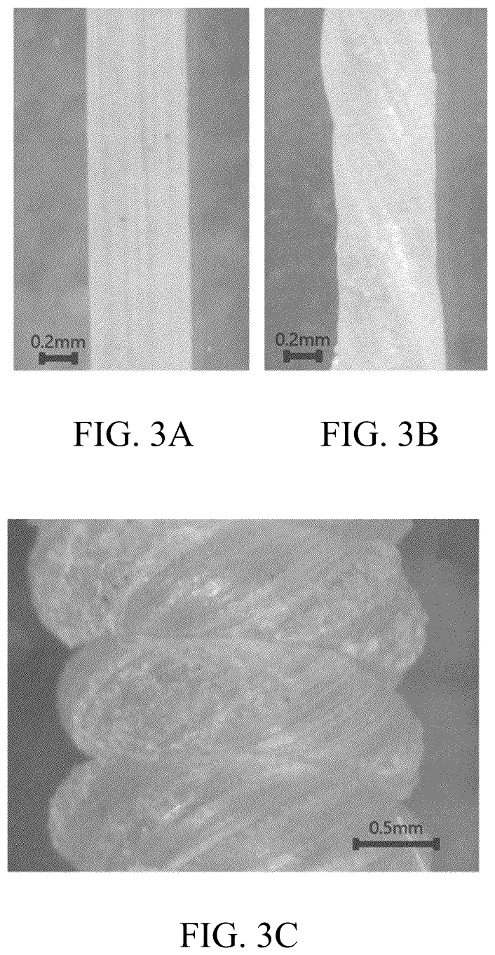

A Mechanical spring is generated from the concept of artificial muscle, which is a material or a device that can demonstrate reversibly contraction, expansion, twisting or bending within one component by applying external stimulus such as temperature, current or voltage. Such actuation mechanism is demonstrated using a nylon fishing wire. Subsequently several tests are done on the springs that made out of precursor fiber (see FIG. 3A) and twisted fiber (see FIG. 3B). It is worth noting that the twisted fiber leads to an increase in axial thermal contraction and recovered torque. The material of nylon 6, 6 fiber is used. The tensile actuation strain is 34% in twisted-then-coiled artificial muscle, which as magnified by 8.5 times than the value in the precursor fiber, as shown in FIG. 3C. After that, a helical SMP spring is provided, and the spring would shrink in length when heated while its diameter would expand. The reason is that the contraction of the fiber length would be compensated by untwisting the filament. It causes the nylon filament to maintain in the same fiber length but have to expand in diameter. This is so called a semi-crystalline structure.

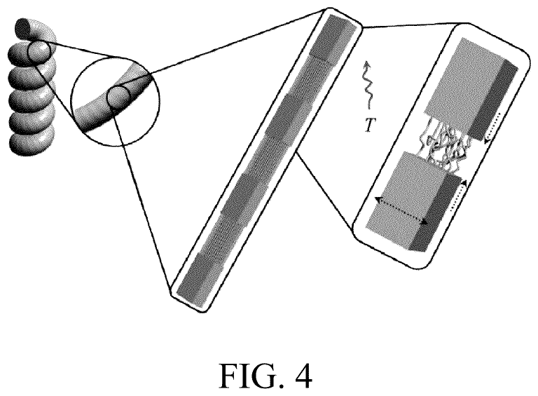

Consequently, the above-mentioned characteristics of the twisted-then coiled nylon spring mechanism are utilized to create the pulling actuator of the present disclosure. When the spring is actuated by heating, it will contract. Once it is cooled, the spring uncoils and returns to its original length. By manipulating the temperature and stabilizing it, the tension can be controlled on each SMP spring. FIG. 4 is a semi-crystalline structure of the polyethylene and nylon.

### Device Operating Principle

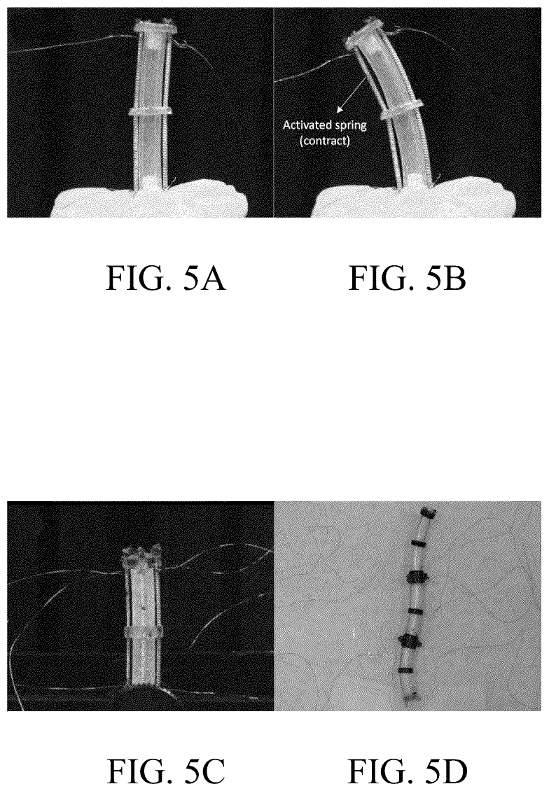

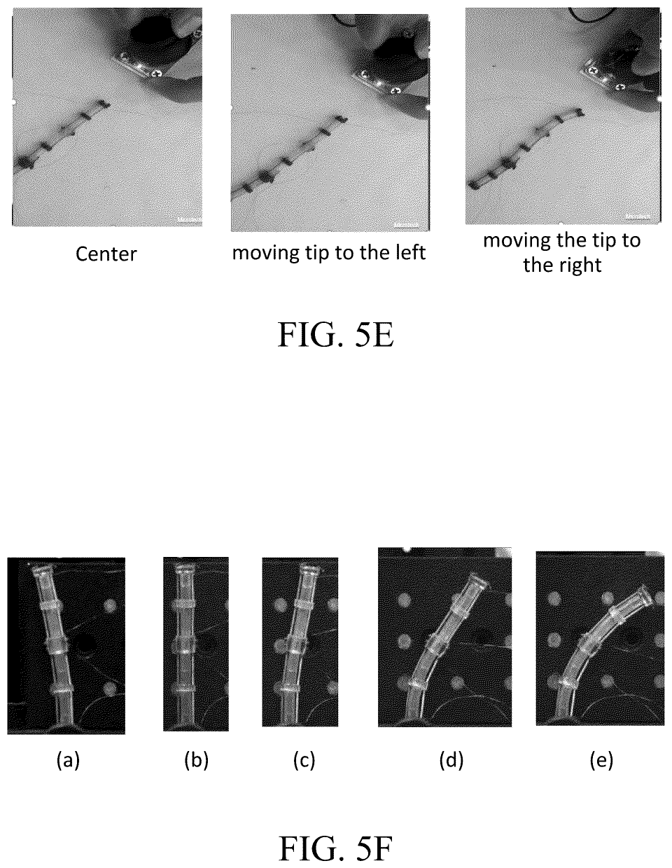

The operating principle of the mechanical spring design is simple but robust. It is a push-pull mechanism that combines multiple springs (see FIG. 5A). When one of the springs is activated and contracted, the contracted one will pull the spring and lead the whole device bend toward its side (see FIG. 5B). The other spring in contrast, it will slightly stretch but the recovery force is smaller than the compression force on the active spring. Using this pulling mechanism, four springs are provided in the present disclosure, as shown in FIG. 5C. Four springs at each direction can create multi-directional movement by adjusting the contraction amount on each spring. This allows movement of our multi-spring actuator to twist, bends in any direction. Moreover, by connecting several of these actuator sections together, the whole device is able to demonstrate more complex snake-like movement (see FIGS. 5D, 5E and 5F).

### Fabrication of Shape Memory Polymer Spring

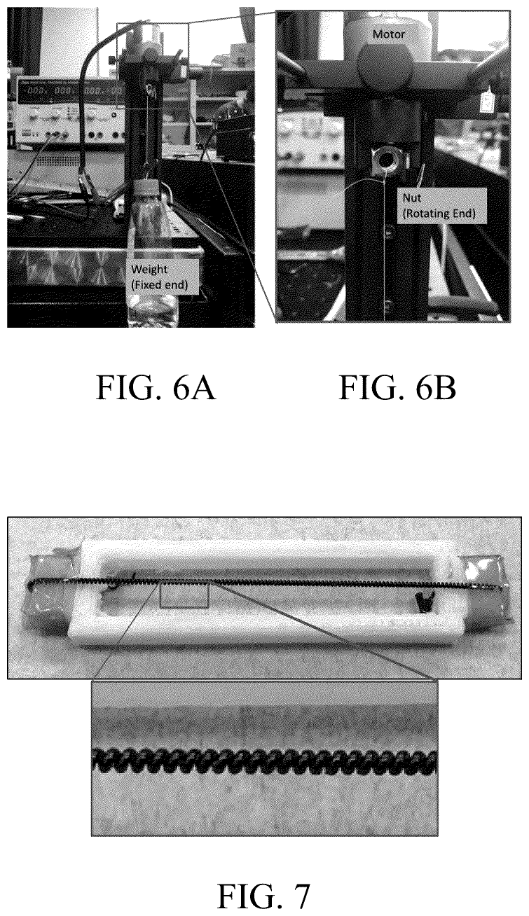

To fabricate the shape memory polymer (SMP) spring, a twisting station is shown in FIGS. 6A and 6B. Nylon fiber, which is the material of our SMP spring, is first secured to screw nut that is fastened to a rotor's shaft of a motor that provided the twist on the fiber. The other end of the fiber is secured to a weight. It provides the tension to the fiber. A tweezer is used and fixed with the weight to serve as a stopper. It would collide with the vertical frame behind and prevent the weight from keep rotating or moving in all directions. However, nylon fiber will not start to coil until the force applied on the fiber reaching certain limit. Again, the hanging weights and the force limits are highly depended on the setup and fiber material properties and dimension. Therefore, series of experiments are conducted to find an empirical model to optimizing the spring fabrication process.

Table 1 shows an example of how some of the control parameters affect the fabrication of a 700 μm diameter spring. The torque motor in this case provides a constant 115 rmp when operating at 12V. Once the spring is completed, it is put into a 70° C. oven, where glass transition temperature of the nylon is around 65° C. to bake for one hour to permanently secure the shape. Purpose of the experiment is to know how tension affects the mechanical performance of the spring. A force-displacement plot with different tension and lengths are tested and summarized after the experiment. More details are discussed later on the performance of these fibers.

TABLE 1
Fabrication parameters of the twisted-then-coiled SMP spring.

| | spring | spring | spring | spring | spring | spring | spring | spring |
|---|---|---|---|---|---|---|---|---|
| | 1 | 2 | 3 | 4 | 5 | 6 | 7 | 8 |
| fiber length | 30 | 30 | 30 | 30 | 30 | 30 | 30 | 30 |
| (cm) | | | | | | | | |
| weight (kg) | 110 | 115 | 130 | 150 | 155 | 165 | 175 | 185 |
| motor Voltage | 12 | 12 | 12 | 12 | 12.005 | 12.005 | 12.081 | 12 |
| (V) | | | | | | | | |
| motor Current | 0.03 | 0.03 | 0.03 | 0.03 | 0.03 | 0.03 | 0.03 | 0.03 |
| (A) | | | | | | | | |
| spring length | 4.396 | 4.139 | 4.556 | 4.615 | 4.725 | 4.881 | 4.843 | 4.826 |
| (cm) | | | | | | | | |

### Electroless Silver Plating Process

Several methods have been developed to heat the SMP spring, such as blowing hot air and flowing through heated liquid. However, these methods are not robust since it is difficult to control the exact temperature on the component. To solve the problem, silver paint coating is used to make the SMP spring conductive and actuated by suing Joule heating. Although the silver paint coating seems to work well, it is not mechanically robust so over time that the coating develops cracks once the spring goes through multiple extensions and contractions. These micro cracks will enlarge over time and render the spring useless. Therefore, the present disclosure develops an electroless silver plating process that not only deposits a thin silver layer on the top of the spring surface but also creates a thin seed layer to enhance the attachment of the conductive silver layer.

The silver plating process requires all samples to be cleaned before the process, because grease, dust, or any particle on the spring surface may affect the quality of the silver deposition. Nylon spring would be immersed into 2.5M sodium hydroxide (NaOH) in a beaker and placed into the ultrasonic washing machine for 5 minutes. NaOH is chosen due to the fact that it can dissolve oil/fat on top of the spring surface. It is then rinsed with DI water to wash away the remaining chemicals and dust residues.

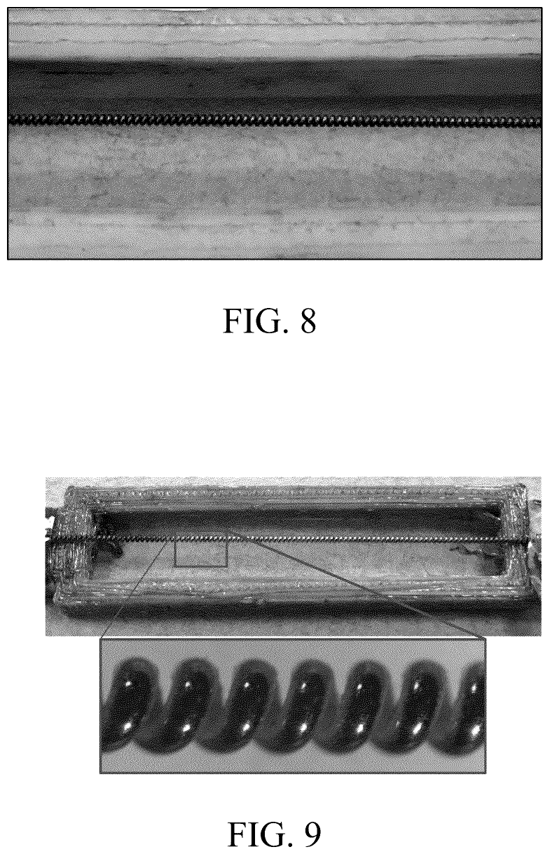

A seed layer is subsequently created by going through an Iodine pretreatment. In this process, nylon spring is stretched a little and fastened onto a frame shape holder by a PI tape. It is then immersed into an Au etchant for 30 minutes under room temperature. The Au etchant is used because it is an I2-KI aqueous solution. During this process, nylon spring will fully absorb iodine complex ions (I2-, I3-) and show a dense brown color (see FIG. 7). Nylon spring is then immersed into 20 wt % AgNO3(aq) for 30 minutes to let iodine ions react with Ag+. Color of the spring would gradually change from brown to a light yellow, which is AgI(s) precipitate. This AgI precipitate stays not only on top of the spring surface but also in the porosity of the spring, which means that it is diffused below the surface of the spring. After that, the spring is placed into 3.7 wt % sodium borohydride (NaBH4), a strong reducing agent, to reduce AgI back to pure silver. Once it is placed into the solution, there will bubbles generated from the spring surface and the color of the spring will change immediately from a light yellow to a dark grey and slowly become a little bright silver (see FIG. 8). At last, DI water is used again to rinse the spring to remove any chemicals and residue out of the surface. Up to this point, a seed layer is provided on the surface of the spring, but it is still not conductive. Next, a silver mirror reaction is needed to create a thicker silver layer on the top of the seed layer.

The silver mirror process begins with mixing 10 wt % AgNO3(aq) and 10 wt % NaOH together in a 20 mL glass bottle with a 5:3 volume ratio. After immersing the spring into the solution, it will immediately generate black Ag2O precipitate. Afterwards, few drops of 33 wt % ammonia are added into the solution until the precipitate exactly disappears. Too much or too less ammonia will cause failure easily. Later on, glucose solution is added to reduce the pure silver out from the silver complex and create a layer of silver on the nylon spring. Chemical formula of the whole process is shown below:

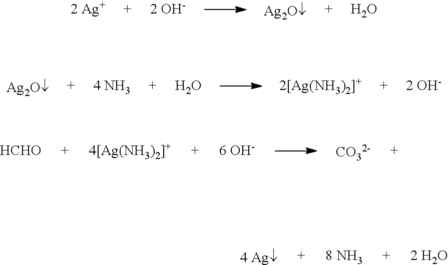

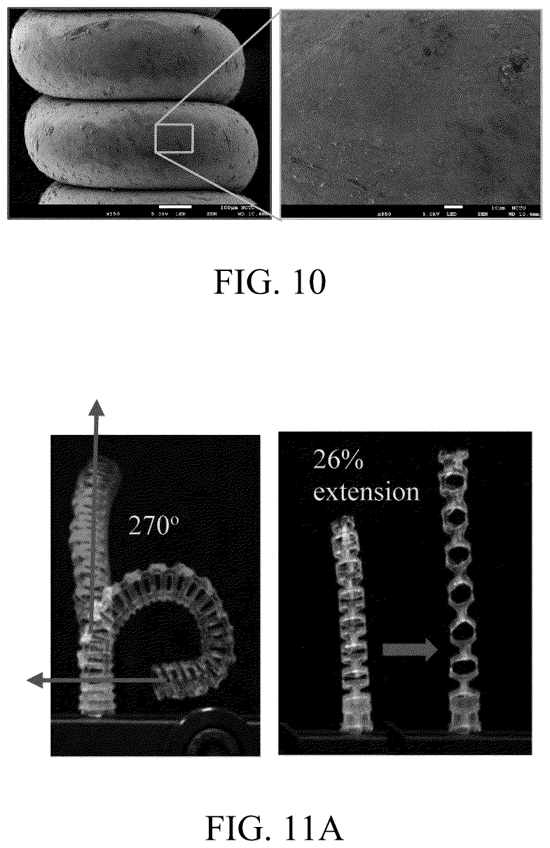

The final Silver-plated nylon spring is shown in FIG. 9. Both optical and microscopic images are shown in FIG. 10, further shows that the process can create a uniform and deep embedding silver electrode on the nylon spring. The silver coating also shows an excellent electric conductivity with a resistivity of 18 Ω/cm. Subsequent load tests will further validate the electrical and mechanical robustness of this sliver plating process.

### Second Embodiment

### Tubular Actuator Design

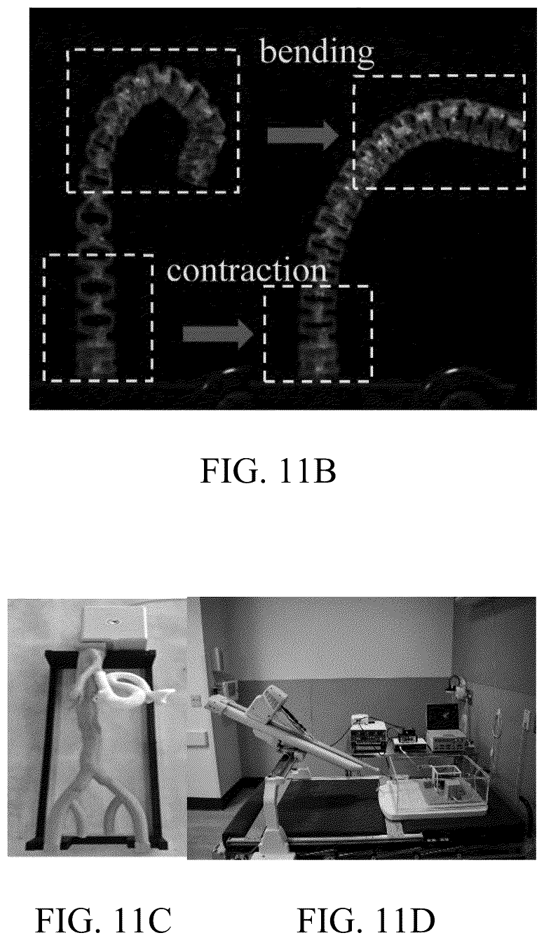

In the second embodiment of the present disclosure, an all-tubular actuator catheter utilizes a single continuous tubular shape actuator (as shown in FIG. 11A) to generate the bending, extension, and contraction (as shown in FIG. 11B), and a combination of bending and contraction (as shown in FIG. 11C) by appropriately activating an area or combinations of areas with electro thermal energy. Certain optimized negative Poisson ratio structure and PU based shape memory polymer are developed and used to accomplish the combination of the actuations.

For the all tubular actuator design, the design will utilize perforated structure similar to the ones shown in FIGS. 11A-11D with a PU-based shape memory polymer to generate the multidirectional bending plus axial extension and contraction (e.g. >270° pitch raw yawn bending, rotation and >30% extension and contraction).

The catheter geometry and dimensions of the designs are determined through modelling and optimization using ANSYS numerical software. The specific parameters are studied and optimized as follows.
1. Shape memory material selection with an emphasis on producing the largest force actuation, fastest actuation speed, minimal electrical and mechanical hysteresis, and longest life cycles.
2. Selection of conducive coating agents to ensure good electrical conduction and adhesion to the shape-shifting polymer, based on our successful silver chemical plating process or carbon nanotube composite.
3. Actuator dimensions: For the exoskeletal spring catheter design, <500 micron diameter for a spring to fit inside the sidewall of the catheter tube. For the all-tubular catheter, singe tubular design with perforated or mesh structure, with tube size relative to the catheter diameter.
4. Maximal bending angle for each catheter design, examining material selections and mechanical designs to minimize the input power.
5. Tip and contact forces between the catheter and the sample: To prevent tissue damage during actuation, tip stiffness and 3-point load tests will be performed on the catheters. The initial specification will be based on the stiffness and 3-point load measurements performed on the steerable tip sections of 5 commercially available catheter systems. For applications in electrophysiology, physician indicates that a force under 10 grams between the tip and tissue compromises the effectiveness of radio frequency ablation, with an optimal force range of 10-20 g. 40+g of force risks perforation. Some tissue sample tests will be performed with the prototype self-sensing catheters to fine tune the resistance force with an appropriate haptic feedback on our controller system.

### Sensor

The actuator is also an inductance and capacitor, with varying inductance and capacitance due to bending (or extension or contraction or any physical movement) or temperature changes in the spring (see FIGS. 2D and 2E). Because of this built-in sensing capability, the precise tip position and rotation can be found through careful calibration with known applied loads (temperature or bending) for each actuator array segment. The normal resistance change versus displacement of the spring also measured (see FIG. 2F). However, it can clearly be seen that resistance value doesn't respond to the change in displacement as well as inductance. The curve “saturates” in this example when displacement reaches 0.4 mm and beyond. After 0.4 mm, even the displacement is increasing, resistance didn't change much.

The inductance and capacitance detection circuit uses a LC circuit with an input range of 10⁻⁶-10 uH and 0.1 pH or 10⁻⁶-10 uF and 0.1 pF sensitivity to prevent interference caused by contact and temperature fluctuation of the electrical contacts (see FIG. 1). If more channels are used, a multi-channel 24-Bit Capacitance-to-Digital Converter will replace the analog LC circuit to increase sampling speed and sensitivity (±4 pH or fF).

With these self-sensing features, they can also be used to create a close-loop feedback system to prevent the tip of the catheter from damaging the walls of the blood vessel. With this feedback, catheter insertion can be made safer and more reliable, thereby reducing risk of injury to patients.

### Fabrication Process of Patterned Shape Memory Tube

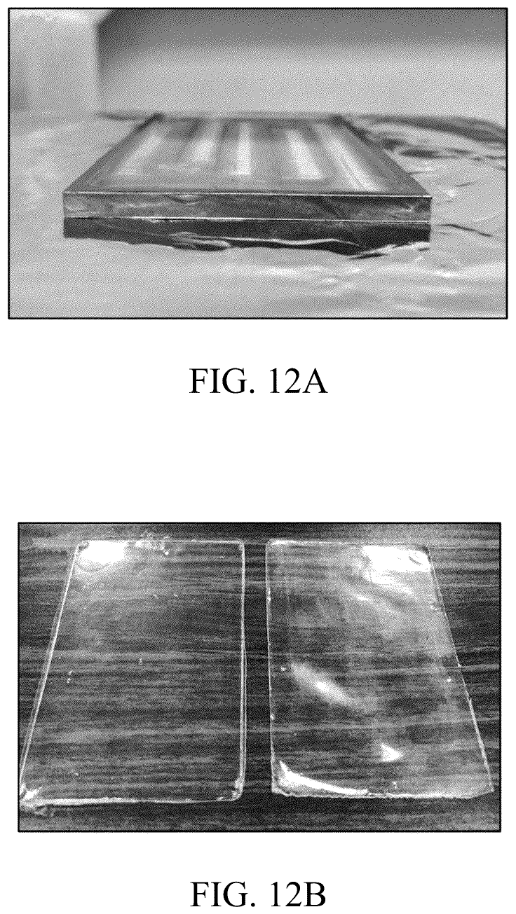

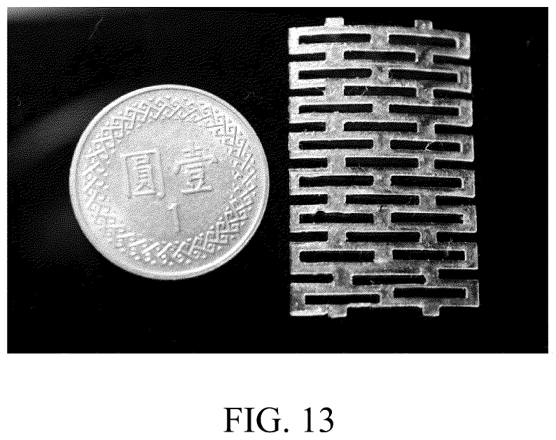

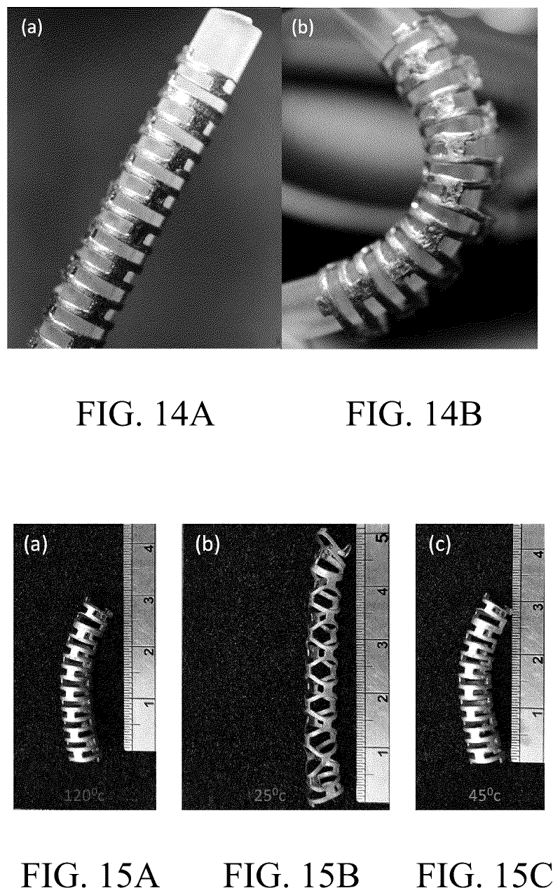

The fabrication of the patterned tube starts from a sheet. The material is MS-4520, a polyurethane-based shape memory polymer solution from SMP Technologies Inc. Solution is first diluted to 60% weight ratio by Dimethylformamide (DMF). Next, it is poured into a rectangular aluminum mold and shear casted to a 2.5 mm thickness sheet by a blade. Pre-dry is done by heating the mold to 80° C. and hold it at that temperature for 10 minutes and followed by the curing process which rise the temperature to 140° C. and keep it for 4 hours. FIG. 12A shows the photos of SMP during curing process and the 400 μm sheet removed from the mold after it is fully cured. Subsequently, the rectangular window array pattern on the SMP sheet is cut by using a CO₂ laser (current set to 26 mA) (see FIG. 12B). Afterwards, a conductive layer will be deposited on the surface of the sheet to achieve the possibility of joule heating. After the metal deposition, the SMP sheet is rolled into a tube and fused together using a soldering iron (see FIG. 13). Moreover, FIG. 14A shows a straight configuration of a rectangular window patterned tube, and FIG. 14B shows a bent configuration of the rectangular window patterned tube.

### Programming the Tube

The SMP tube can be programmed into any shape. For the shape memory polymer, MS4520, Tₚₑᵣₘₐₙₑₙₜ is around 120° C. and the glass transition temperature, Tg, is 45° C. To program the permanent shape, the tube is held to desired shape and placed in an oven with a 120° C. temperature for 20 minutes (FIG. 15A). It is then cooled to room temperature naturally. To create the temporary shape, an external force is then applied at any temperature less than 120° C. The actuator can stay in this shape even if the external force is removed (FIG. 15B). Finally, once the tubular actuator is reheated above 45° C., it will automatically go back to the permanent shape (FIG. 15C), the tube can be reprogrammed if it is re-heated above 120° C.

### Third Embodiment

### Carbon Nanotube Composite Patterned Tube

In order to improve the SMP patterned tube, an electrical controllable, carbon nanotube (CNT) composite SMP is provided. The background, fabrication and electrical and mechanical characterization are described as follows. The multidirectional movement is demonstrated. Additionally, different methods to achieve reversible movement are also provided.

### Fabrication Process of Carbon Nanotube (CNT) Composite SMP Patterned Tube

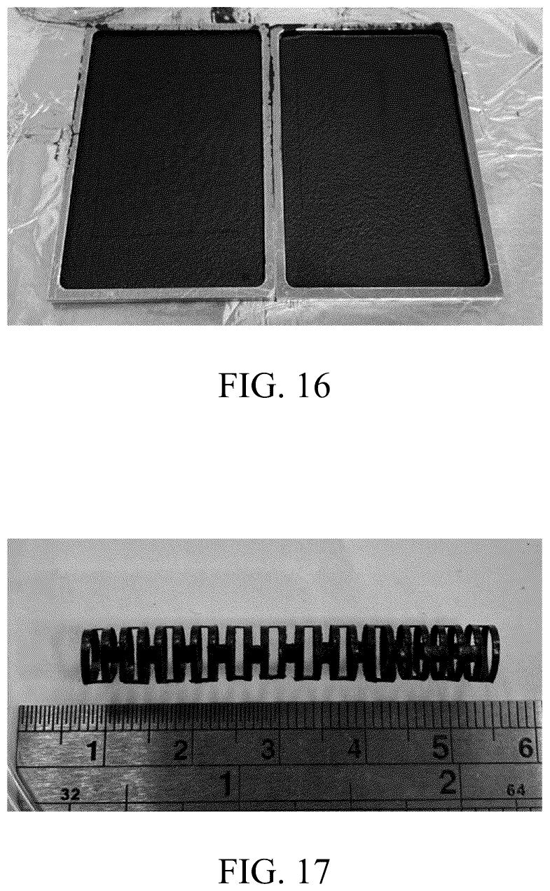

Fabrication process of the CNT-based SMP patterned tube is actually the same as the process of previous pure SMP tube (Second Embodiment) but using different solution. In the present disclosure, the CNT-based SMP solution is made by mixing liquid phase SMP, DMF, and CNT powders together with a weight ratio is 40 wt %, 59.35 wt %, and 0.65 wt % respectively. Due to CNT increases the viscosity of the solution, 2 hours of stirring time is needed to ensure CNT is uniformly dispersed. Afterwards, the mixed solution is shear casted using the same 2.5 mm deep aluminum mold followed by a 2 hours curing process (FIG. 16). A 220 μm thick CNT-based SMP sheet is the product. It is then patterned with the CO₂ laser but with a higher current power which is 29 mA (26 mA is enough for pure SMP). Finally, it is rolled up into a patterned tube with 45 mm in length and 6 mm outer diameter (FIG. 17).

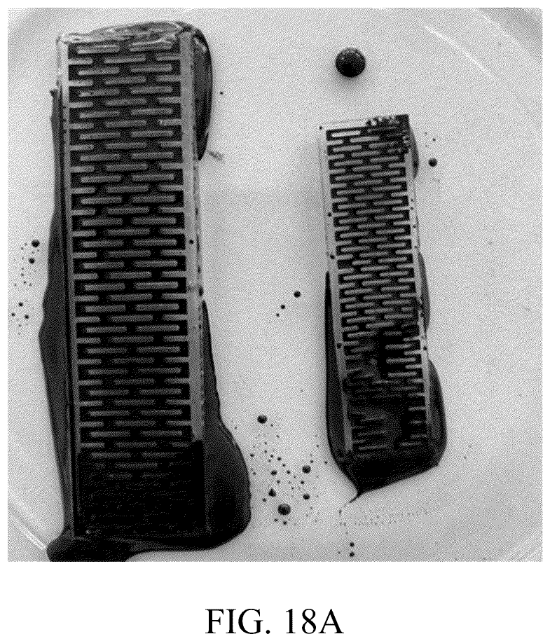

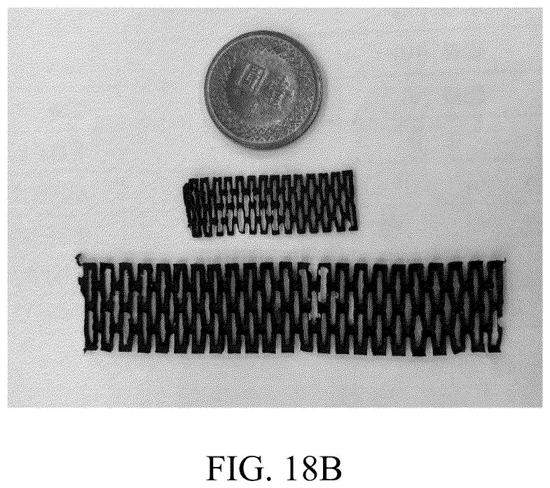

Another technique to fabricate the spring is using the molding process. First, a hard ABS mold is printed using a 3D printer. Then, a PDMS mold is replicated from the master and use as the primary mold to make the final product as shown in FIGS. 18A and 18B.

### Electrical and Mechanical Property of CNT-based SMP

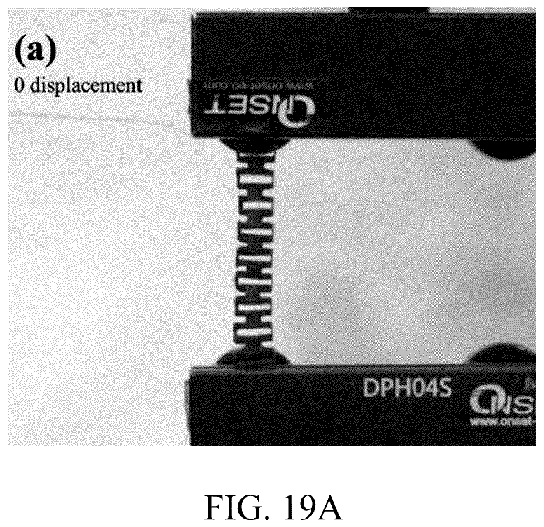

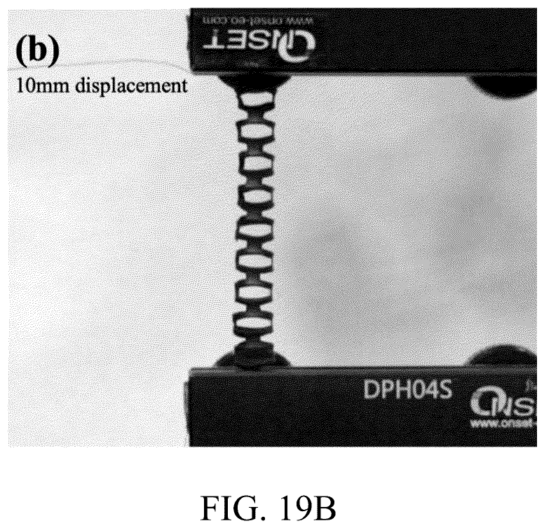

To compare stiffness and electrical property with the pure SMP patterned tube, the same load test is also conducted on the CNT-based SMP tube with the same setup. Both ends of the tube is clamped on the stage and the testing specimen is 35 mm long originally. Maximum stretching ratio is also set the same as previous tests at 28.5%, which means the maximum displacement is 10 mm and the stretching speed is also 1 mm/sec. FIG. 19A shows a stretching load test when the displacement is 0 mm, and FIG. 19B shows a stretching load test when the displacement is 10 mm.

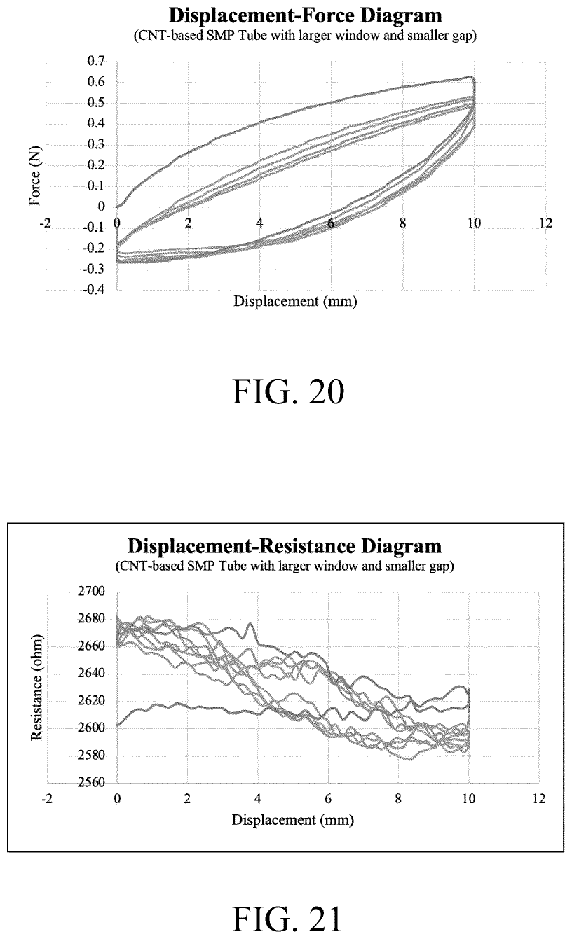

The displacement-force diagram that shows the mechanical property of the CNT-based SMP tube is shown in FIG. 20. From the result, it can be seen that the load cycle is similar to the pure SMP tube, first cycle looks much different than the rest of the cycles. Further analyze the value in the diagram and compare with the result with the pure SMP tube that has the same pattern, force is about 2.5 times smaller. The reason is because wall thickness of the CNT-based SMP tube is 2 times thinner than the pure SMP tube. (Due to concentration of SMP in CNT composite is 20% lower than the pure SMP solution)

Meanwhile, resistance change is also measured when the load test is conducted. Purpose is to compare the electrical property with the pure SMP tube. FIG. 21 shows the displacement-resistance diagram of the CNT-based SMP tube with a larger window and smaller gap pattern. It is obvious that resistance scale of the CNT-based SMP tube is much larger than the pure SMP tube deposited with metal. Although the resistance is much larger, it is more stable, resistance change is only around 100Ω out of 2600Ω (3%) while the metal deposited pure SMP tube is >15%. The reason that CNT-based SMP has this stable resistance is because it is a composite, conductive CNT powder is uniformly distributed in and out through the whole tube. On the other side, metal deposited pure SMP tube only had a thin layer of metal on the outer surface, which is easily to create micro cracks or other defects when the tube is stretched.

### Advantages of CNT-Based SMP

CNT-based SMP is created with the intention to show a more robust design than the other metal deposited SMP designs in terms of its ability to distribute the hear more evenly and holding the heat more steadily during the heating procedure. In the following sections, tests are conducted to validate this hypothesis.

### Heat Distribution on SMP Tube Using Other Metal Deposition Method

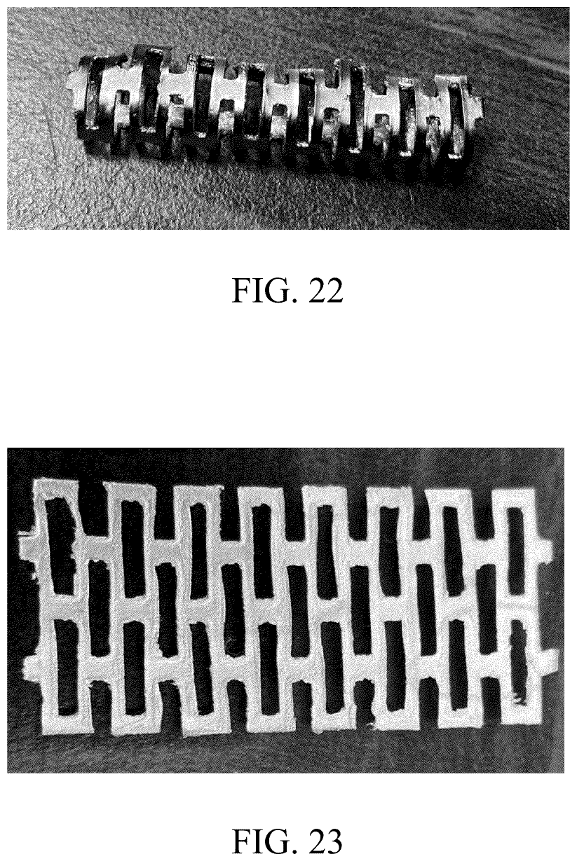

Two different direct metal deposition methods SMPs are tested to see how well the heat are distributed. One is silver sputtered and the other is direct coating of a thin silver expoxy. The first technique uses typical sputtering deposition to form the conductive layer on SMP. After it is rolled and fused together, the jointed parts are sputtered again to ensure surface of the whole tube is conductive. FIG. 22 shows what the tube looks like after second deposition. The electrical resistance of this 5 cm long tube is about 714Ω. Second tube is made from silver epoxy coated SMP sheet. FIG. 23 shows the coated sheet before rolling up. After the sheet is rolled up and more epoxy is applied at the jointed area. This 5 cm long tube resistance is extremely small, 18.1Ω.

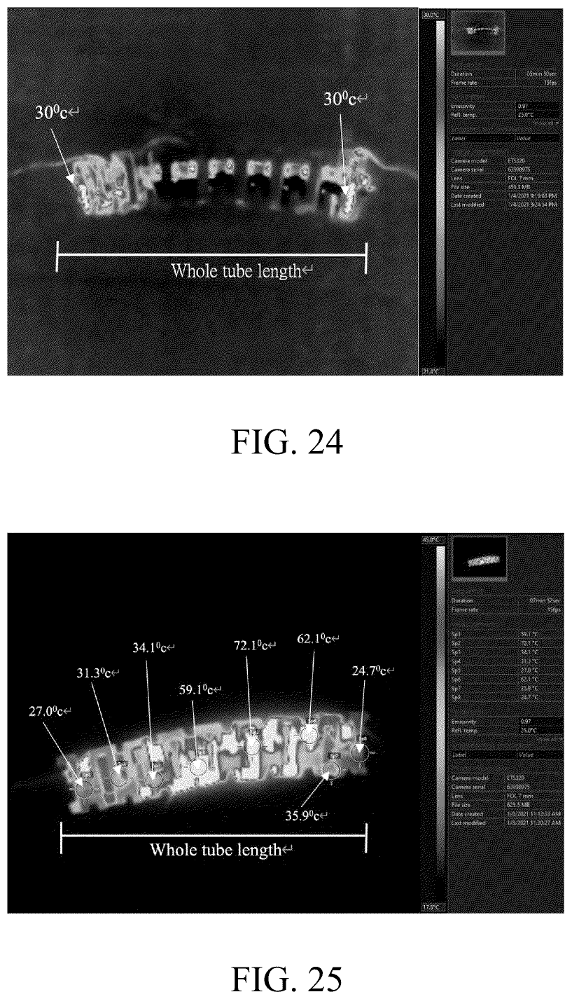

Heat tests are then conducted on both SMP patterned tubes using Joule heating. In these tests, a 375 w high power supply is used in order to provide larger current for the low resistance devices. For the silver sputtered tube, voltage applied is 10V and the current is about 10 mA. FIG. 24 shows heat distribution result under this voltage, only both ends and the jointed part show higher temperature. The reason is because in these parts, resistance tends to be larger so that heat only generated at these areas. It shows that heat distribution on the silver sputtered SMP tube is not uniform.

On the other side, due to SMP patterned tube with silver epoxy has a relatively smaller resistance, it does not need as high voltage. To heat up the tube, there is already 150 mA passing current when 4.8V is applied. Therefore, temperature is much higher across the board (max temp=72.1° C.). Look further into FIG. 25, although heat distribution is more uniform compare to the sputtered SMP, it still only heated up part of the device. The reason of uneven heat distribution is also due to the coating is not uniform and there are many micro cracks or defects formed during fabrication and heating process.

### Heat Distribution on CNT-Based SMP Tube

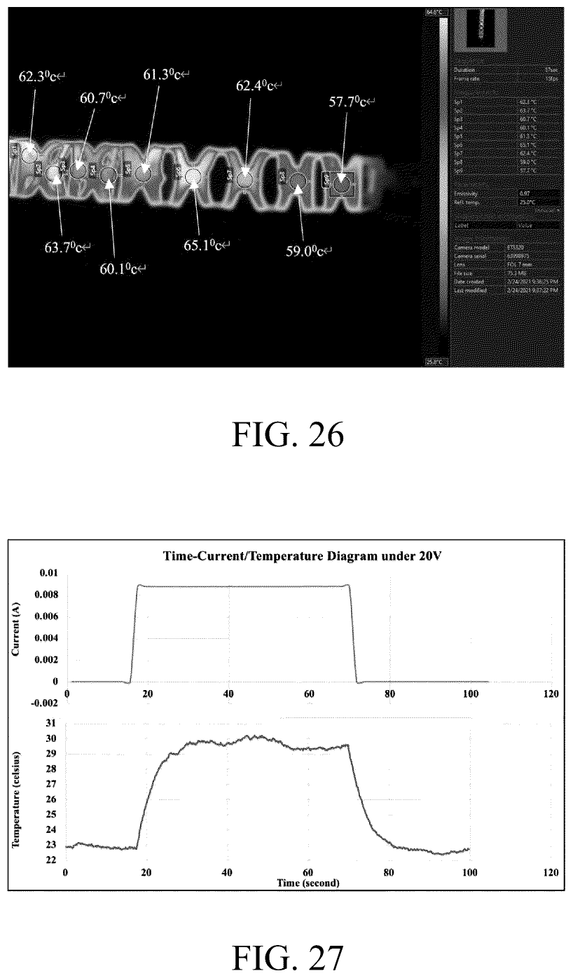

The same heating test is performed on the CNT-based SMP tube. Larger voltage (42V) is used due to larger resistivity of CNT-SMP composite. The current in this case is around 25 mA. As expected, heat distribution is very uniform (see FIG. 26), most of the tube shows temperature around 60° ° C. with minimum temperature variation (±5° C.). This is expected since the composite helps distribute the CNT particles uniformly across the whole SMP: which in terms provide more uniform joule heating throughout the whole SMP not just on the surface like the metal deposited SMPs.

### Temperature Stability During Joule Heating

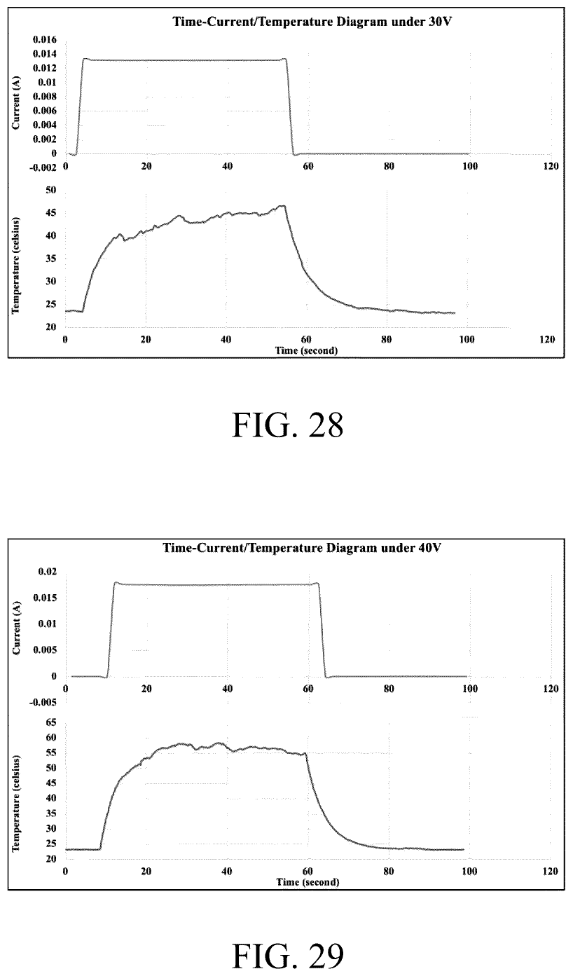

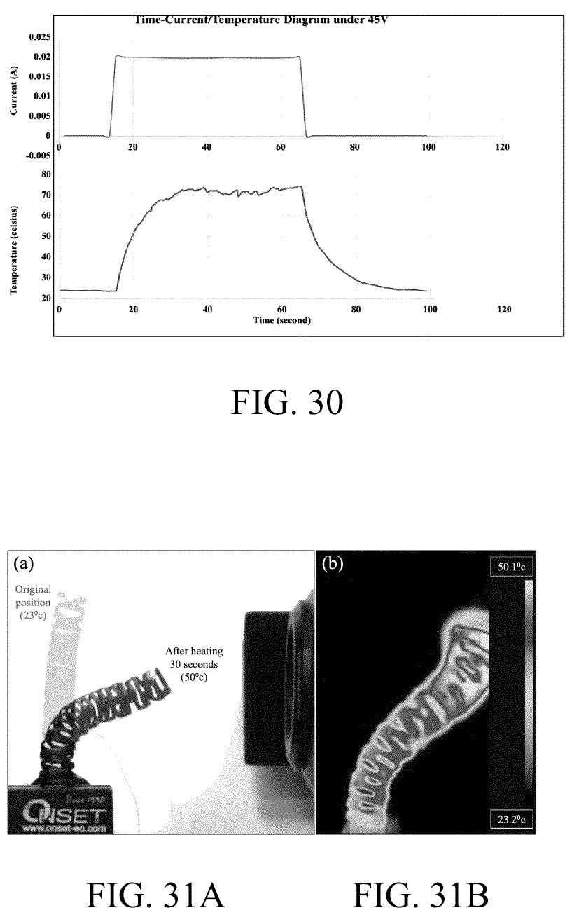

Temperature stability tests are also conducted. Purpose of doing this is to see if the temperature varies during a continuous joule heating. In the test, voltage is kept applying for 60 seconds without any cooling time and the voltage is varied from 20 to 45V. Current and temperature are recorded to see how they varied together. FIGS. 27 to 30 show the time series plot of current and temperature under different applied voltages. These five plots have a similar characteristic: temperature rises to a relatively stable value after 10 seconds after current is applied to the device. Based on the results, it shows higher the voltage, the less stable the temperature is. However, the variation is still within +5° C. The curve also shows it takes a longer time to cool for the higher voltage as expected.

### Multidimensional Movement of CNT-Based SMP Tube

Previous chapters have already demonstrated that SMP patterned tube can do several actuations, including extending, contracting, and bending. By improving the electrical property using CNT-based SMP, more complicate movement such as twisting or even locally control can be achieved. In this section, two movements are demonstrated.

### Twisting Actuation

In the twisting actuation test, the CNT-based SMP patterned tube is used with the pattern that has larger window and smaller gap because the structure is more flexible than the other. Length of the tube is 45 mm and the outer diameter is 6 mm. The twisting shape is programmed by putting the shaped tube into a 120° ° C. convection oven and bake for 1 hour. Afterwards, it is then cooled down to 60° ° C. and an external force is applied to define straight temporary shape. For the actuation process, initially 30V is applied and the device temperature reached about 50° C. Under this temperature, tube began to twist but the twisting speed started to slow down after 30 seconds. FIG. 31A shows the overlay pictures taken at the beginning and after voltage is applied for 30 seconds. FIG. 31B shows the temperature distribution of the device.

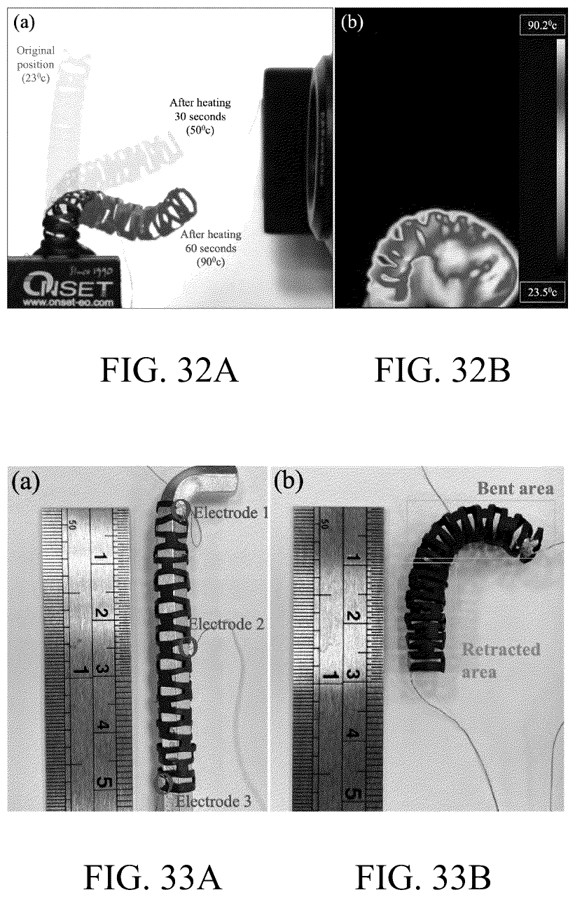

To further increased the twisting speed and also checked the ultimate twisting performance, 45V is applied which temperature increased to 90° C. FIG. 32A shows that the tube further twisted then turned and let the tip pointed upward. From this twisting actuation test, it proves that CNT composite SMP tube can demonstrate very complex movement and can provide multi degree of freedom with ease. FIG. 32B shows a temperature distribution on the CNT-based SMP tube after applying 45V.

### Partially Control for Heating Specific Part

Partially control technique that activate only part of the device can further create unique motions and widen the device application. Testing device with 50 mm length, 6 mm outer diameter and 220 μm wall thickness is used. Permanent shape is defined as this straight configuration. To partially control the device, three electrodes are provided, two electrodes at both ends and one electrode at the middle which it would be grounded as shown in FIG. 33A. This creates two separate parts to be controlled by the electrical inputs. Resistances are also measured: upper part is 0.926 k Ω and the lower part is 0.892 kΩ. It allowed us to control either part when we apply voltage to upper or lower electrode. Afterwards, the temporary shape is defined by using an external force, lower part is contracted to shorter length of which the contraction ratio is 25% and the upper part is bent to around 120° (see FIG. 33B). It is expected that when we activate lower part, it would extend back to original length. On the other hand, the upper part would be bent upward when it is activated.

Three tests are conducted. First, the upper section is heated to 60° C. and then cooled. Second, the lower section is heated to 60° C. and then cooled. Lastly, the upper section is heated again but increased to 90° ° C.

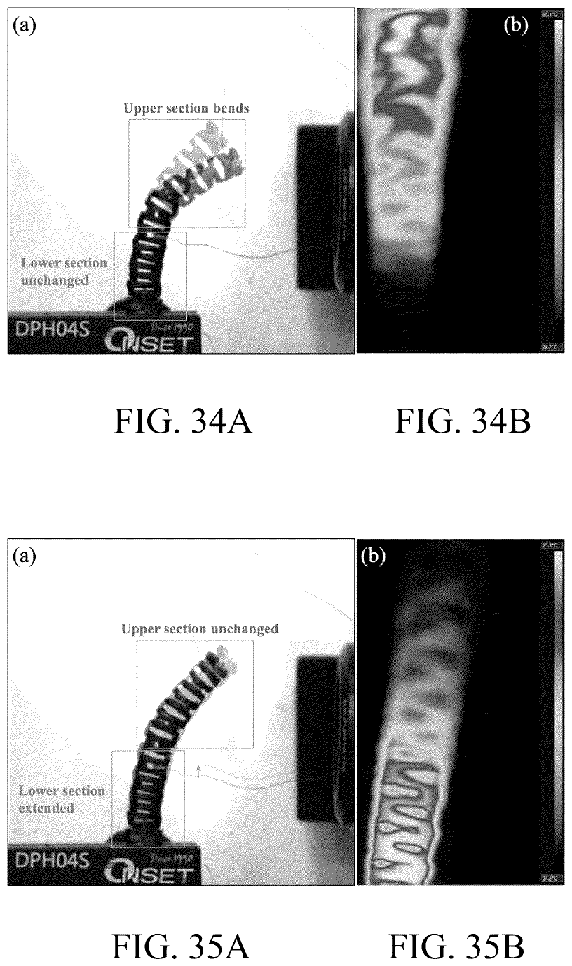

In the first test, 13.9V and 0.01 A is applied to the upper tube. FIG. 34A shows that heat only generated at the upper part while lower part remained at room temperature. The upper move about 25 degrees and the lower area stayed still (see FIG. 34B).

Next the lower part is heated, voltage and current are 13.9V and 0.01 A respectively. Under this operating condition, temperature of the lower part also rose to 60° ° C. and started to extend back to the original length but not apparent movement in the upper section (see FIGS. 35A and 35B).

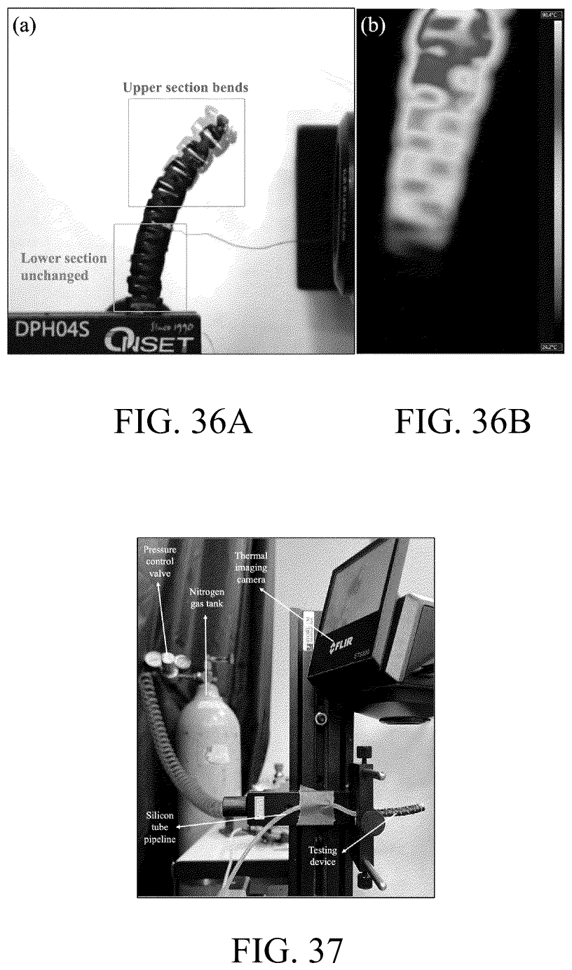

In the third test, the upper section is reheated with a higher voltage (20V) and) (90° ° C. FIG. 36A shows the temperature distribution and also the shape recovery result. Upper section further bent 10 more degrees back due to the higher temperature and length of the lower section remained unchanged just as expected. FIG. 36B shows a temperature distribution of the whole tube when only applied 20 V to the upper section of the tube.

### Reversible CNT-Based SMP Tube

Due to the characteristic of most of the shape memory polymer is a one-way actuation, it only recovers to the pre-defined permanent shape when thermally activated. Except using an external force, it will not deform back into the previous temporary shape even when temperature cools down lower than its glass transition temperature (Tg). Thus, in the present disclosure, different methods (including bi-layer and pneumatic processes) and the so-called two-way actuation are presented as follows to create the reversible movement.

### Pneumatic Method/Process

Since the shape memory polymer is soft when thermally activated but stiff when in room temperature, method that offer soft and flexible support when the device is heated but become stiff when device cool down is required. In this study, a pneumatic technique is developed. The idea and assembly procedure is the same as the dual tube, the thinner silicone tube that has 2 mm inner diameter and 3 mm outer diameter is used as a support, and connected to a nitrogen gas tank with a pressure control valve. The CNT-based SMP tube is 42 mm long, 5.5 mm outer diameter with 220 μm wall thickness, and has a 1.598 kΩ resistance. The pattern that is used on the tube is the one with a smaller window and a larger gap, and is the one with a stiffer structure because it would bend more when activated.

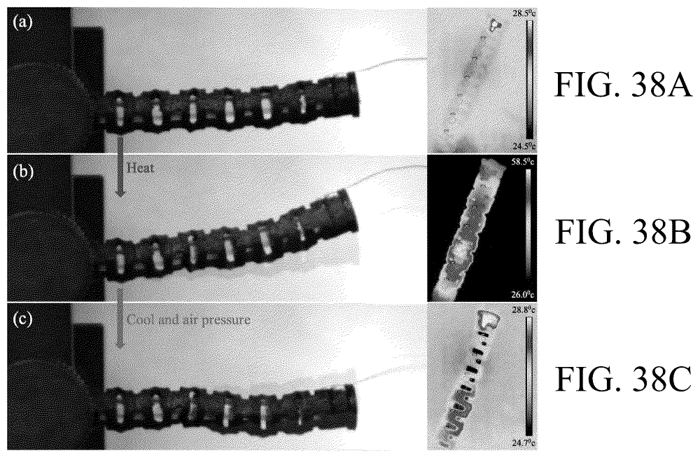

A testing setup is shown in FIG. 37, a FLIR camera and a CCD camera are used to capture images. A 30V and 18.643 mA input are used to heat up the SMP tube to around 55° C. When the device is heated, no gas is released so the thin silicone remained really soft. Therefore, the CNT-based SMP patterned tube could be bent upward easily as shown in FIG. 37. After bending angle reached the limit, power supply is turned off and nitrogen gas is released to enhance the stiffness of the silicone tube. Air pressure is gradually increased from 0 to 30 psi and the whole device started to bend downward. It is because when high pressure air flew through the silicone tube, it forces the inner tube to become stiffer and straight. FIGS. 38A to 38C show that the device totally bent back to original straight configuration. Specifically, FIG. 38A shows an original straight temporary shape of the CNT-based SMP tube, FIG. 38B shows the CNT-based SMP tube started to bend after heated upon 58.5° C., and FIG. 38C shows the CNT-based SMP tube bent downward when voltage is set to 0 and started to add air pressure. From the thermal imaging, it can be seen that nitrogen gas also gives advantage of a faster cooling speed.

### LC Polymer and Bi-Layer SMP Structure

The reversible shape memory polymer has higher elastic modulus when the temperature is lower than its Tg. Thus, after thermally activated and recover to the permanent shape, it become stiff when cooled down. If we wish to reverse it back to the temporary shape again, an external force is required. To overcome this, the two-phase SMP material can be used, such as reversible LC elastomer or creating a reversible bi-layer structure such as combining PI layer to assist the recovery.

The later design involves both material and mechanical design modification to enhance this reversibility. Although the SMP pattern tube has excellent shape memory characteristics, it can only perform one-time memory effect. Therefore, in order to create a reversible electroactive actuator, a polyimide (PI) film is combined with a SMP film. Form a composite material with PI/SMP bilayer structure to realize reversible driving. When heating the entire device, the molecular chain of the SMP film containing crystalline and amorphous sections will become disordered and soft when the temperature rises. While heating, since the SMP film and the PI film are directly attached, the contact is excellent, and the heat can be transferred to the PI film with almost no loss. In the heat dissipation process of the shape memory effect, both the SMP film and the PI film will shrink in volume. The coefficient of thermal expansion (CTE) of PI at room temperature (CTE) is 2.8×10-5 K-1, which is lower than SMP. Therefore, although SMP dominates the shape memory effect, because the shrinkage rate of SMP is relatively large, PI plays an extremely important role in the process of shape reversible recovery.

### CNT-SMP/PI Bi-Layer Design

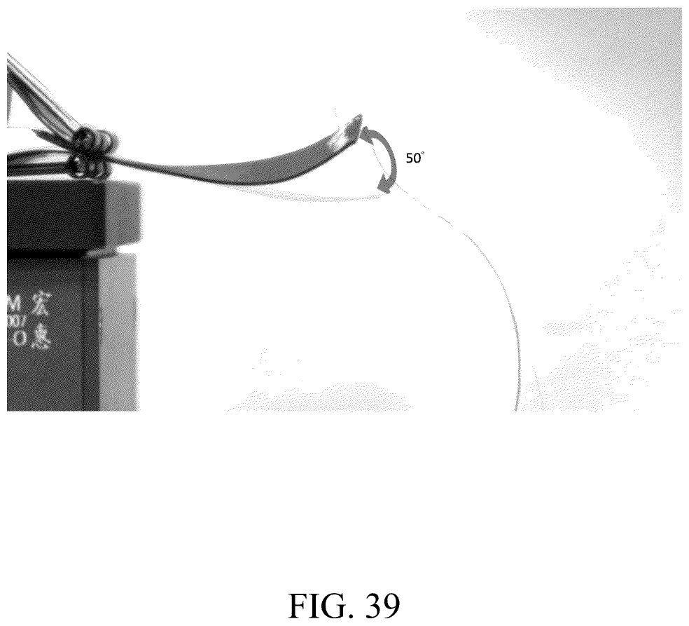

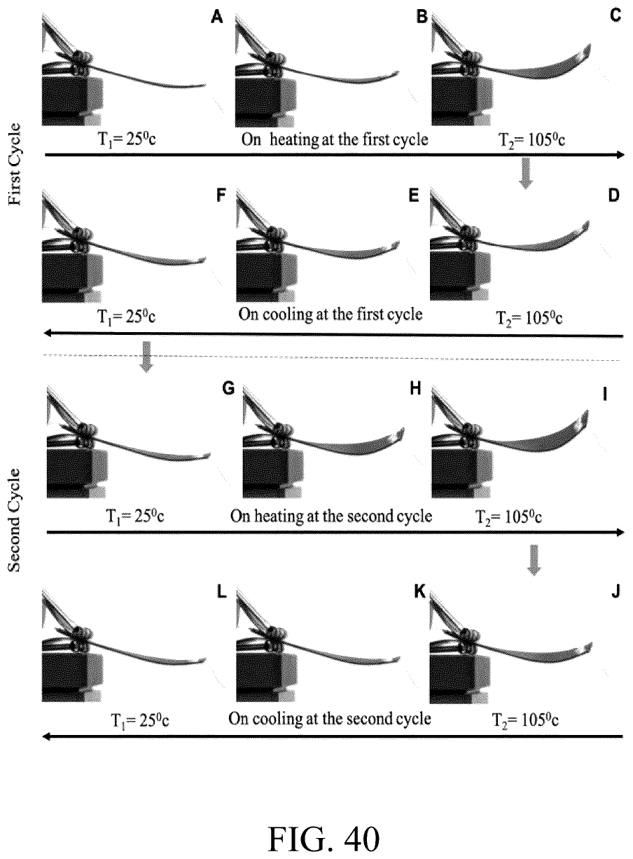

The bi-layer helps recover the SMP actuation due to their difference in thermal expansion and thickness. For the later, the optimal design has a thickness ratio of 1:5=PI: CNT-SMP, which generates a 50° bending angle, as shown in FIG. 39. The two-way shape memory behavior in the CNT-SMP/PI bilayer beam actuator can be seen in FIG. 40. This show a typical shape memory polymer behavior where smaller bending after the first cycle, but one can clearly see with this new design, a two-way behavior (reversible) can be achieved.

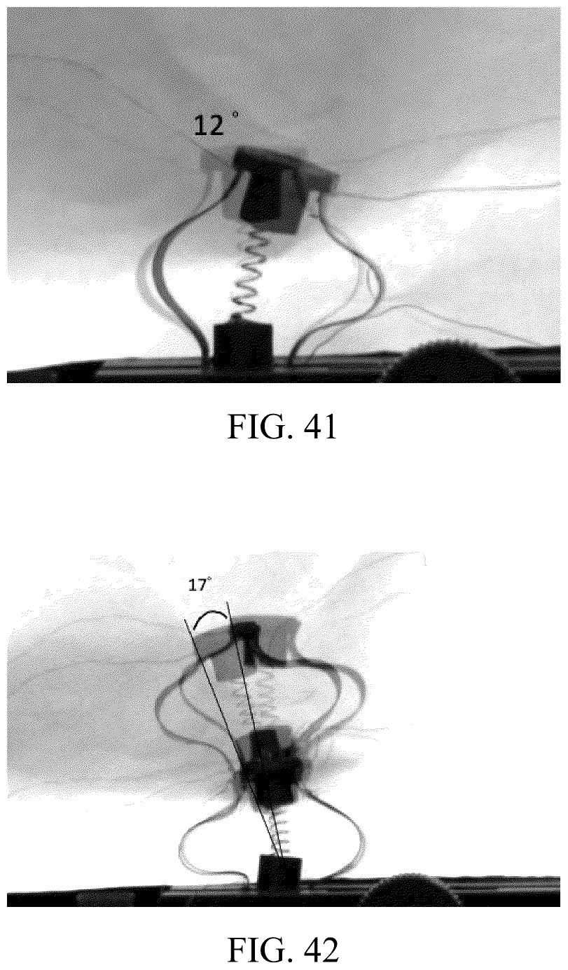

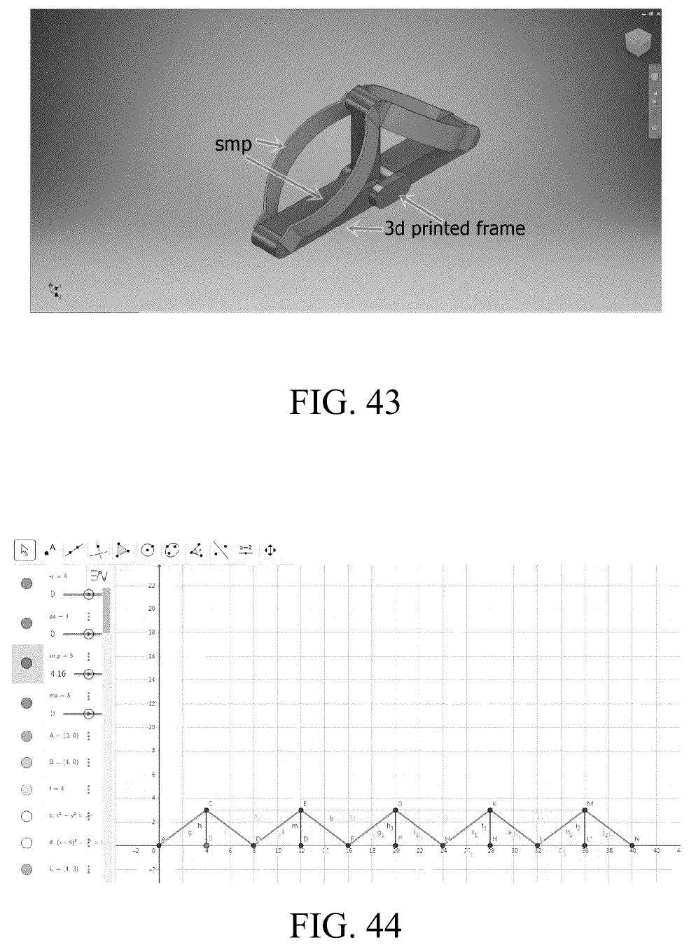

FIG. 41 shows an example of a bending or multi-DOF actuator made of two of these bi-layer cantilever strips where the two-way actuation can be achieved with on or multistage actuator design. FIG. 42 shows that the bending or multi-DOF actuator is achieved by implementing these bi-layer CNT-SMP/PI strips to generate large two-way (i.e., reversible) bending or extension motion.

### Linkage Design

A linkage structure is a combination of the mechanism structure and bi-layer CNT-SMP/PI 2-way actuator. The dual SMP at the same hypotenuse is to help increase the bending angle.

Moreover, the maximum bending is simulated in GeoGeBra to under how it moves when SMP start to shrink. As shown in FIG. 44, red is SMP and yellow is the boundary wall of the tube and black represents the 3D printed structure.

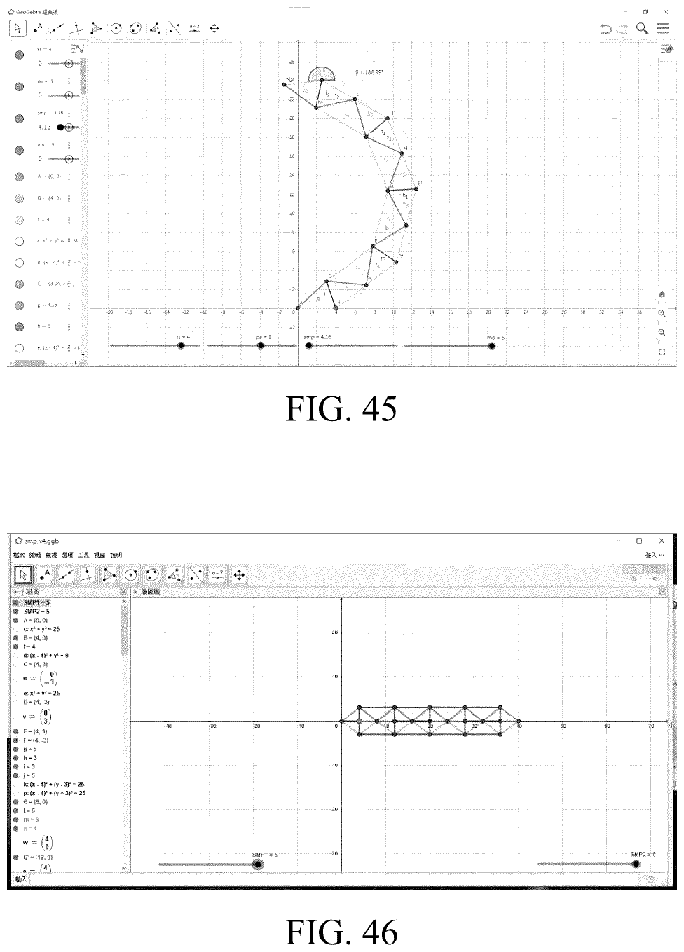

Further, in the simulations, as shown in FIG. 45, 5 segments of linkages are used and 187 degrees bending is successfully achieved. The shrinking percentage of the SMP is at 83%. The bending result is shown in FIG. 45.

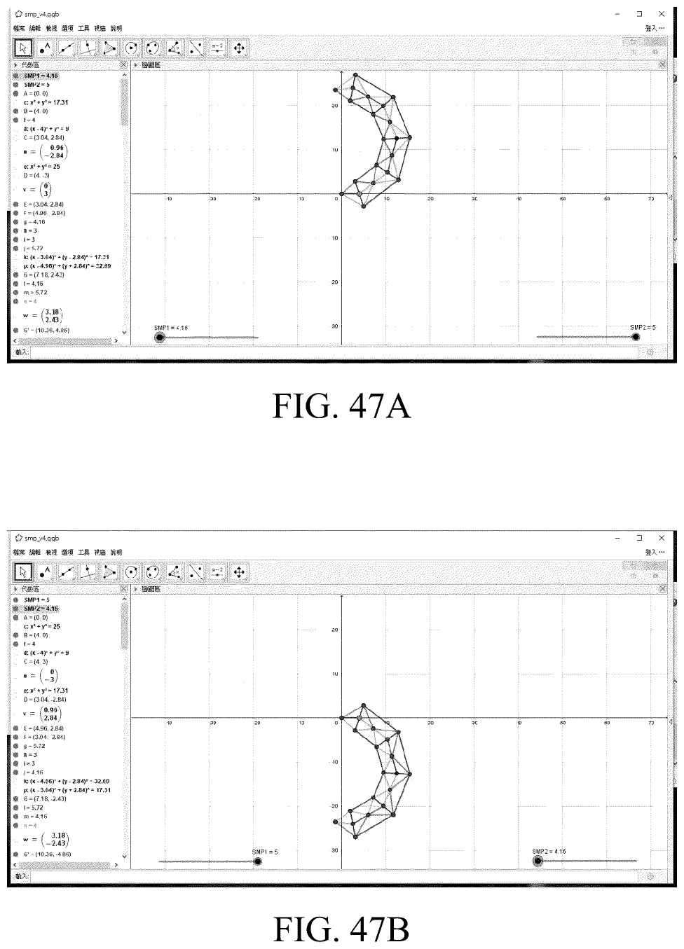

In addition, a reversible SMP linkage design is also provided by using GeoGeBra in the present disclosure, as shown in FIG. 46. FIG. 47A shows a maximum upper bending angle of the reversible SMP linkage design, and FIG. 47B shows a maximum lower bending angle of the reversible SMP linkage design.

### Fabrication

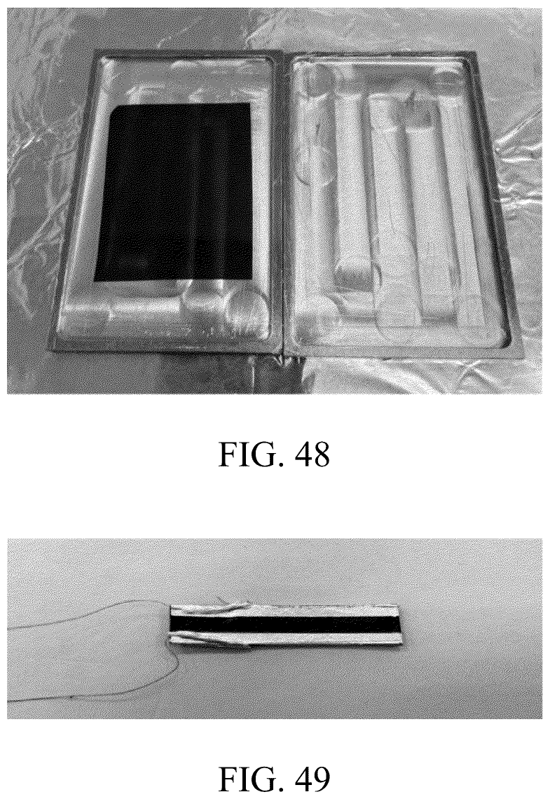

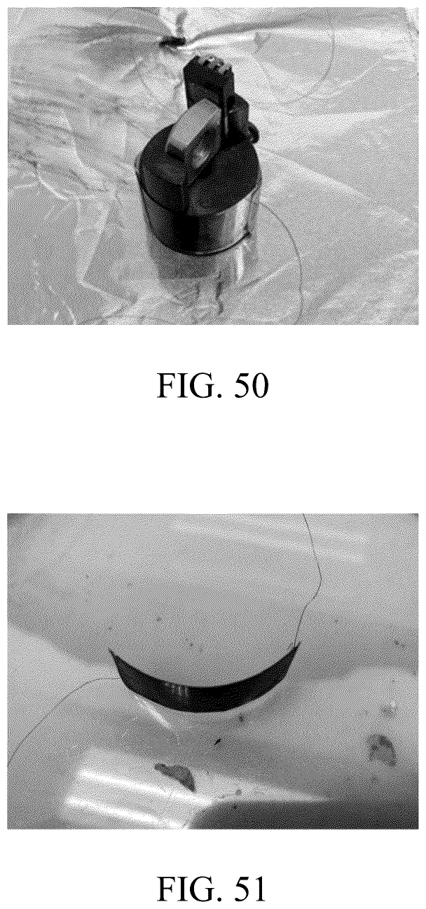

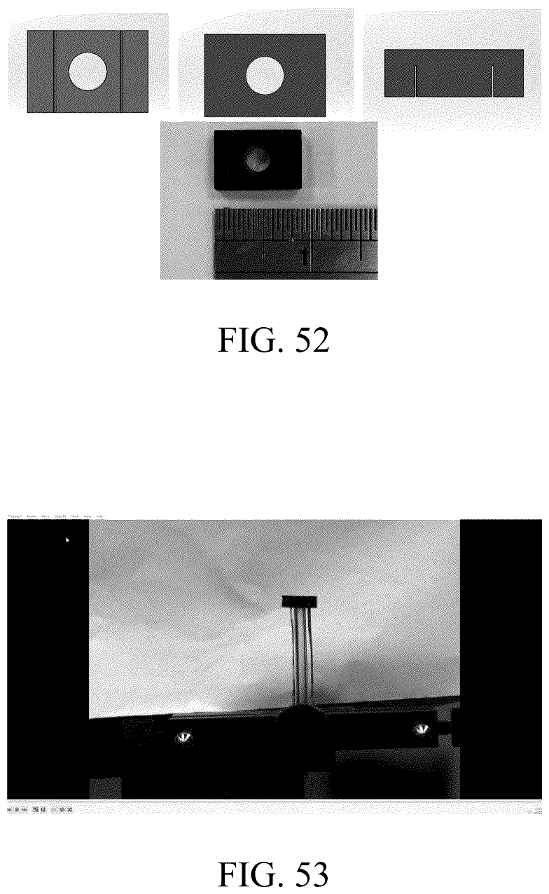

1. Put the PI film and CNT-SMP into the mold, then do shear casted with glass slide, and use a hot plate to bake it at 140° C. for 4 hours. The thickness of bilayer structure is 415 μm (75 μm PI film+340 SMP film). FIG. 48 shows that the PI film and CNT-based SMP is put into a mold, and shear casted with a glass slide.
2. Cut the film into the same size 6 mm×30 mm, and use silver glue to fix both ends of the test piece to fix the copper wire. FIG. 49 shows the film is cut into the same size, and silver glue is used to fix both ends of the test piece to fix the copper wire.
3. Fix the film on a metal cylinder and heat it in an oven at 140 degrees for 5 minutes, the purpose is to form a curved permanent shape. FIG. 50 shows that the film is fixed on a metal cylinder, and heated in an oven at 140 degrees for 5 minutes. FIG. 51 shows that a curved permanent shape is formed.
4. Cool the film to room temperature. Then put it back in the 50° C. oven to soften the film, and quickly press it back into a flat shape (temporary shape) after taking it out of the oven. It will be shaped again when the temperature drops back to normal.
5. Then fix the two test pieces on a holder with a width of 6 mm, a length of 8.8 mm, and a height of 3 mm. The middle of the holder is a hole with a diameter of 2.8 mm to hold a silicone tube with an outer diameter of 3 mm and an inner diameter of 2 mm. FIG. 52 shows a dimension of a holder.

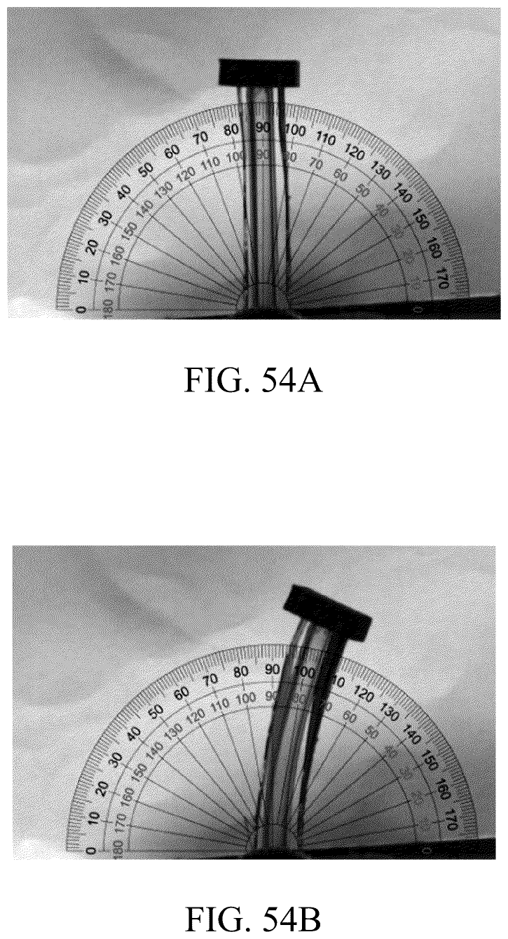

FIG. 53 shows a testing setup for the tube. 0.87A is applied to the test piece on the left and do 4 cycles. It can be seen that after the first cycle, the silicone tube cannot return to its original position, but in the next few cycles, it can return to the initial position of the second cycle. The most important thing is that the bending degree is about 10°, and it can be reverse to temporary shape (flat shape) by cooling the test piece. FIG. 54A shows a tube testing before heating. FIG. 54B shows a tube testing after heating.

The actuation is further improved by reducing the axial and transverse direction stiffness of the actuator design so the tube can easily bend and extend or contract more easily in the actuation directions. One of the ways is to incorporate auxetic structure in the design to reduce the stiffness of support but also bring much larger deformation behavior. Although the design produces negative Poisson's ratio. The whole purpose is to utilize the existing large deformation design that is already incorporated in these designs for our catheter design.

### Auxetic Design

In order to expand the overall mechanical performance of the tubular actuator design, many mechanical designs are explored. Among the many meshing designs, there is a series of unique structures called negative Poisson's ratio structure which not only can reduce the stiffness of support but also bring superior deformation behavior.

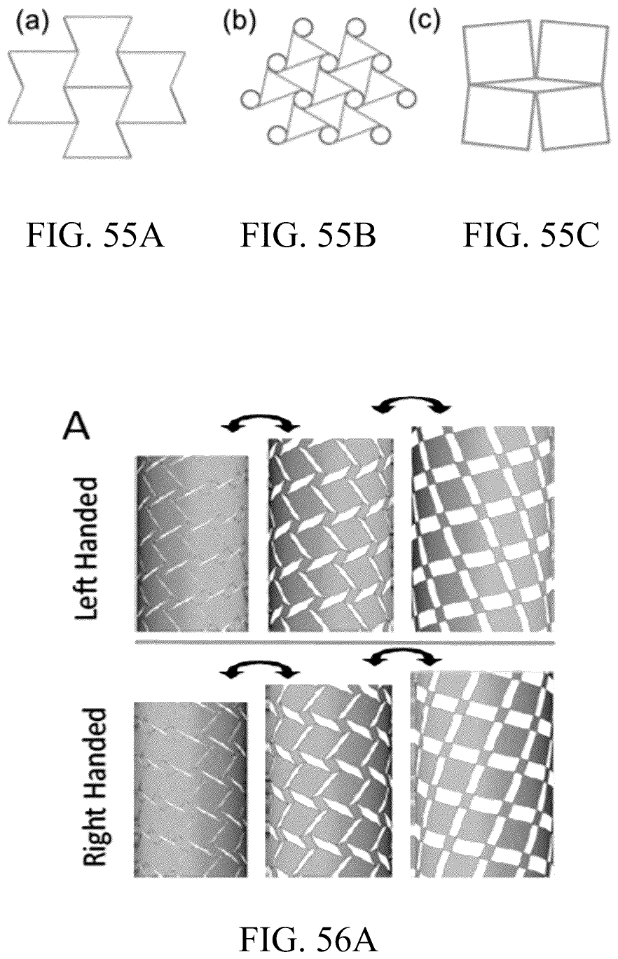

The NPR structures can be classified into three common types: re-entrant honeycomb, chiral honeycomb structure and rotating rectangles, as shown in FIGS. 55A to 55C. That is, FIG. 55A shows a 2D re-entrant honeycomb structure. FIG. 55B shows a 2D chiral honeycomb structure. FIG. 55C shows a 2D rotating rectangles structure.

However, according to the mechanism to show the NPR behavior, they can also be divided into two categories: by folding (see FIG. 55A) or rotating (see FIGS. 55B and 55C).

After comparison, since the 2D chiral honeycomb structure is too complicated to fabricate, only rotating rectangles and chiral honeycomb structure prototypes are investigated.

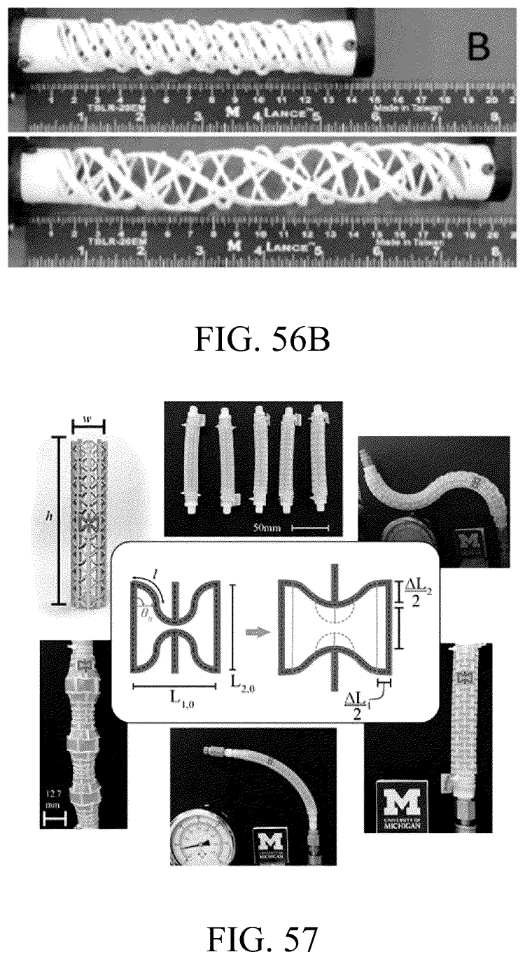

About the NPR structure used in an actuator, a kind of actuator is provided based on the rotating rectangles structure (see FIGS. 56A and 56B).

On the other hand, the sleeve with NPR structure is presented based on the 2D re-entrant honeycomb structure. From FIG. 57, it shows the superior bending behavior. Thus, this NPR structure is chosen to combine with HAP device.

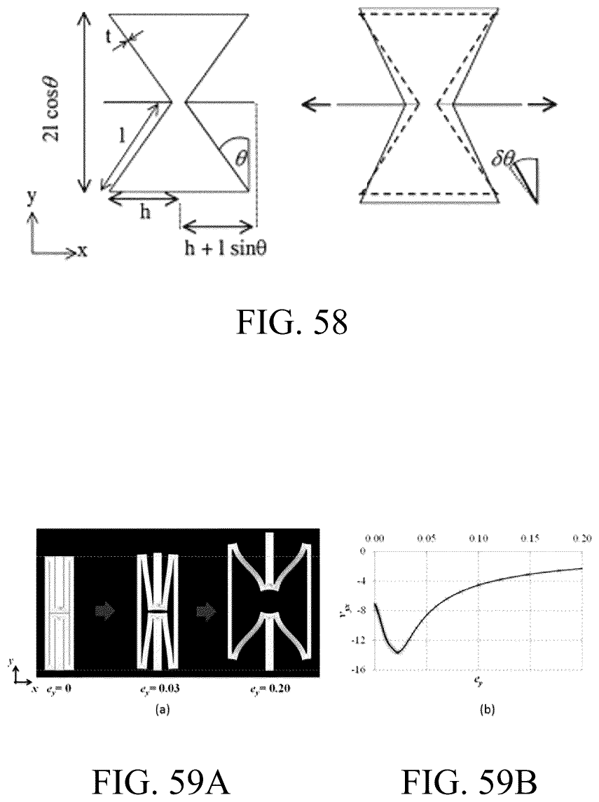

FIG. 58 shows basic parameters in 2D re-entrant honeycomb geometry, particularly illustrating the behavior of conventional and auxetic honeycombs and foams. The Poisson's ratio and Young's modulus along the loading direction are presented as below.

$${v}_{12} = \frac{sin θ ( h / l + sin θ )}{{cos}^{2} θ} \qquad ( 1 )$$

$${E}_{1} = k \frac{( h / l + sin θ )}{b {cos}^{3} θ} \qquad ( 2 )$$

$$k = \text{?} {b ( \frac{\text{?}}{l} )}^{3} \qquad ( 3 )$$

$$\text{?} \text{indicates text missing or illegible when filed}$$

where h, l, b, θ are as defined in FIG. 58, and Es is the intrinsic Young's modulus of the material forming the cell walls. n is Poisson's ratio, and k is the stiffness.

In addition, FIG. 59A shows the 2D re-entrant honeycomb structure at different degrees of strain in the y direction, and FIG. 59B shows that Poisson's ratio changes with the degrees of strain in the y direction.

FIGS. 55A to 59B are for illustration only and can be found in existing documents.

### Fabrications

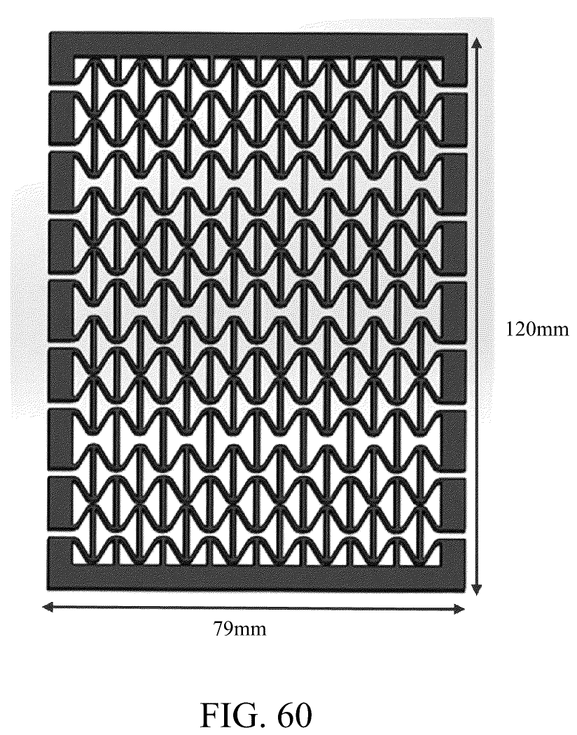

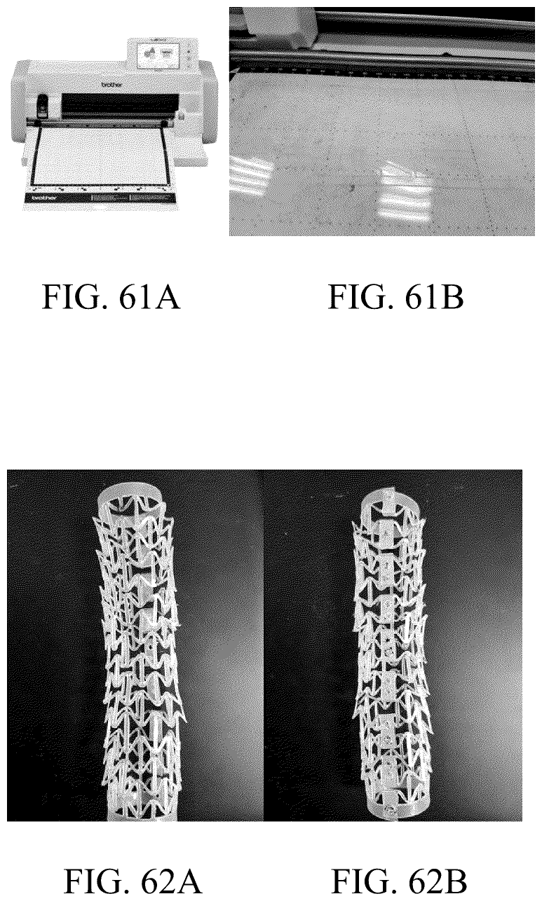

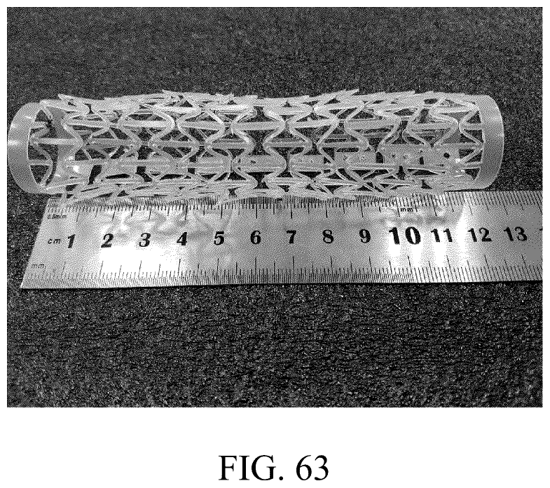

To combine 2D re-entrant honeycomb structure with the heat-activated polymer. HAP sleeve design is introduced (see FIG. 60A). The 2D re-entrant honeycomb structure is designed using the SolidWorks. and then the pattern is transferred onto the HAP sheet (see FIG. 60B) by stencil cutting machine (see FIG. 61A). Finally, the HAP 2D re-entrant honeycomb sheet directly and connect two sides of it by heat melt (as shown in FIGS. 62A, 62B and 63).

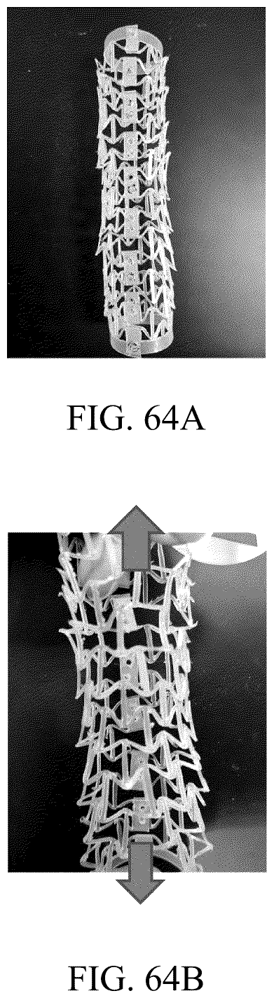

Although HAP sleeve shows NPR behavior successfully (see FIG. 64A), the scale of it is too huge to be activated by heating wire 1 use. It is needed to be scaled down. However, the minimum line width produced by our stencil cutting machine is only achieved to 1 mm. Thus, 3D printer is used to print out the HAP sleeve device for better result.

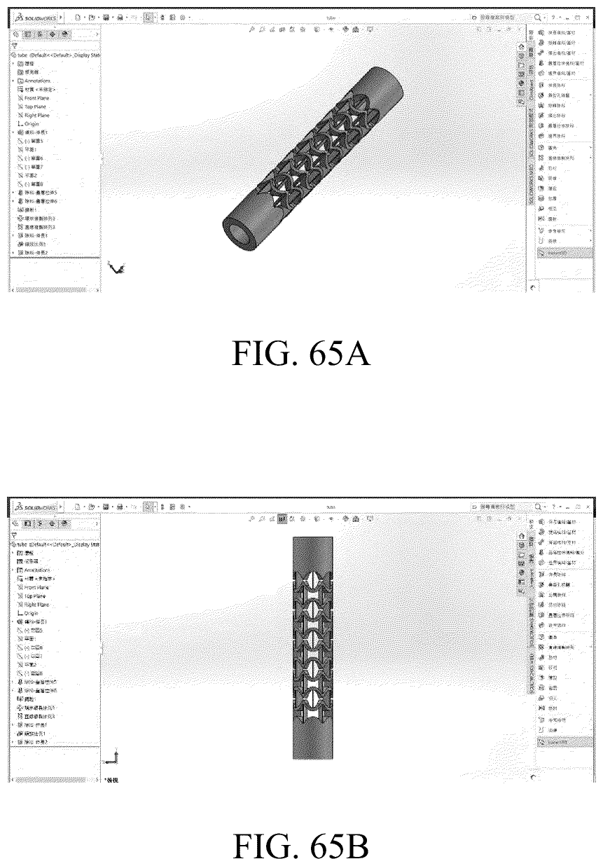

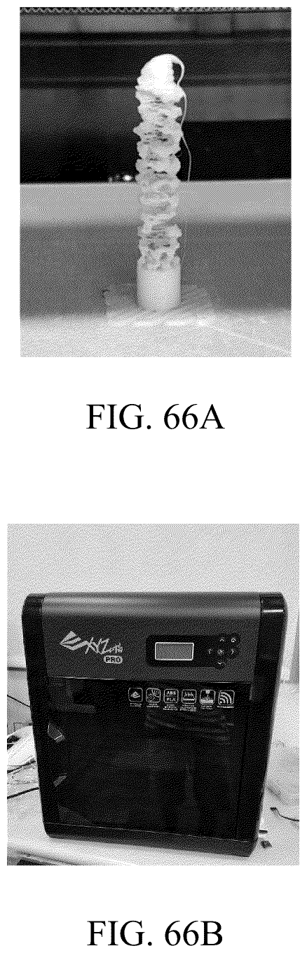

The design of tubular NPR actuator (see FIGS. 65A and 65B) is referred to as the HAP sleeve. For printing, the model is first designed in SolidWorks and then the generated STL. File is sent to 3D printer to generate the final prototype (as shown in FIGS. 66A and 66B).

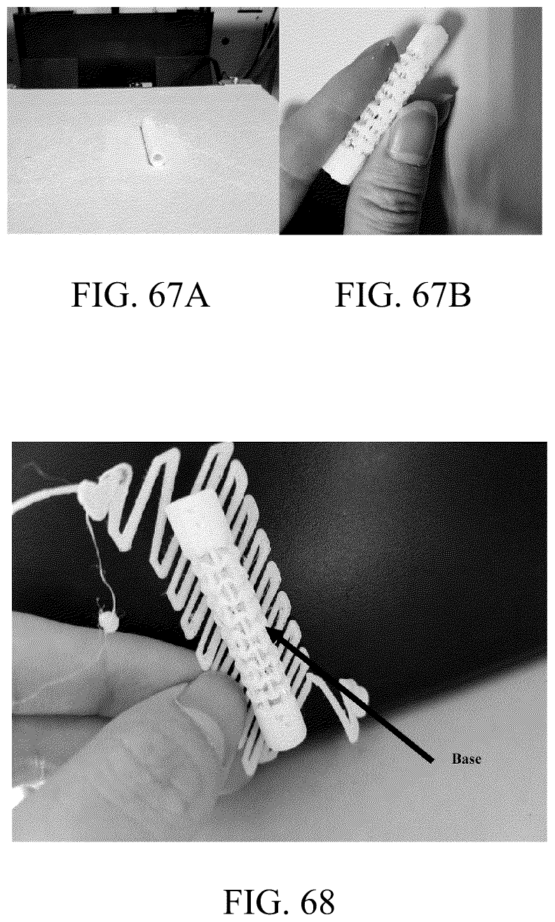

From FIG. 66A, it shows bad result to print along axial direction. Thus, it is printed along transverse direction this time. Although HAP is piled up at the bottom side, the result becomes better (see FIGS. 67A and 67B). FIG. 68 shows that the tubular NPR actuator is printed on the base.

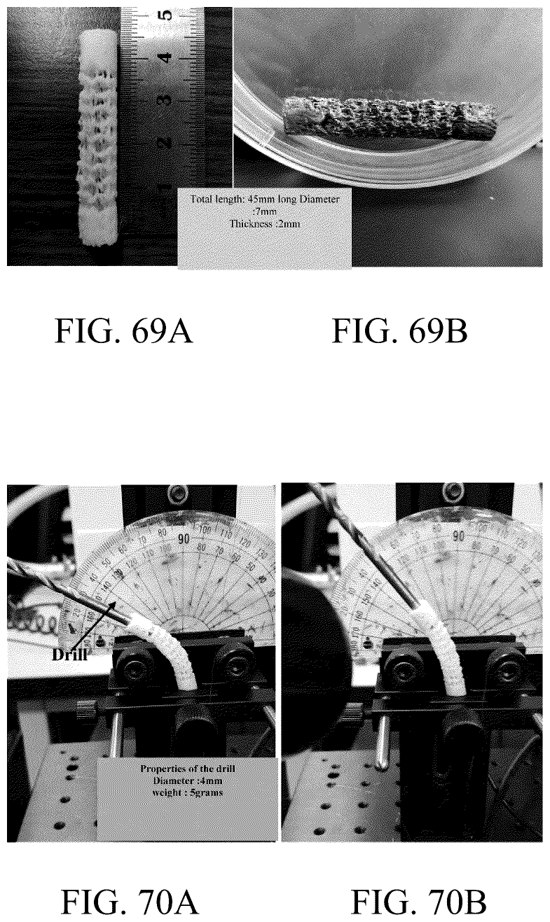

FIG. 69A is the best tubular NPR actuator result at present although lots of HAP threads hung between the gap is observed. After the electroless plating process (as shown in FIG. 69B), it shows that a stable conductive layer is attached to its surface successfully.

### Mechanical Performance

The tubular NPR actuator is pre-stressed before heating as shown in FIG. 70A. A drill is put inside the actuator as the loading. After heating it up, the bending angle is shown about 20 degrees (see FIG. 70B).

The reason why the tubular NPR actuator not only shows large bending angle but also provide strong output enough to pick up the loading seems is because there is lots of HAP thread exists between the gap.

Furthermore, from a manufacturing perspective, HAP is also printed by a printer through rapid cooling down process just like the quenching process. Once again, it is confirmed that quenching process tends to make heat-activated polymer become more active.

Other rapid prototyping techniques similar to the previous disclosed shear casting and also spin coating and other thin sheet fabrication can be applied in creating these 3D actuator from sheet configuration.

### Strain Enhancement Design

The strain enhancement is increased based on the reducing Poisson's ratio (ex/ey) and localized strain. Mechanically, the strain measurement can be enhanced by modifying the strain gage structure. Several geometries and dimensions that are ideal to enhance the strain measurement have been found.

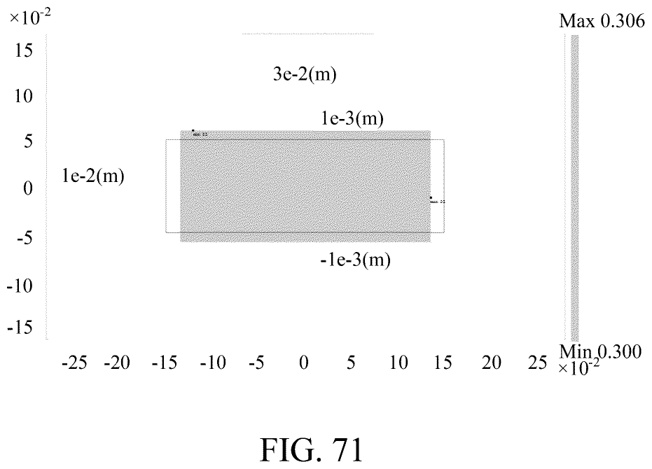

In FIG. 71, a uniform piece of rectangular patch is shown. If 20% strain is given to the rectangular structure from top to bottom, it is expected that 20% (1st principle strain) would be the same everywhere in the structure. The Poisson's ratio is expected to be Poisson's ratio=0.33=−ex/ey.

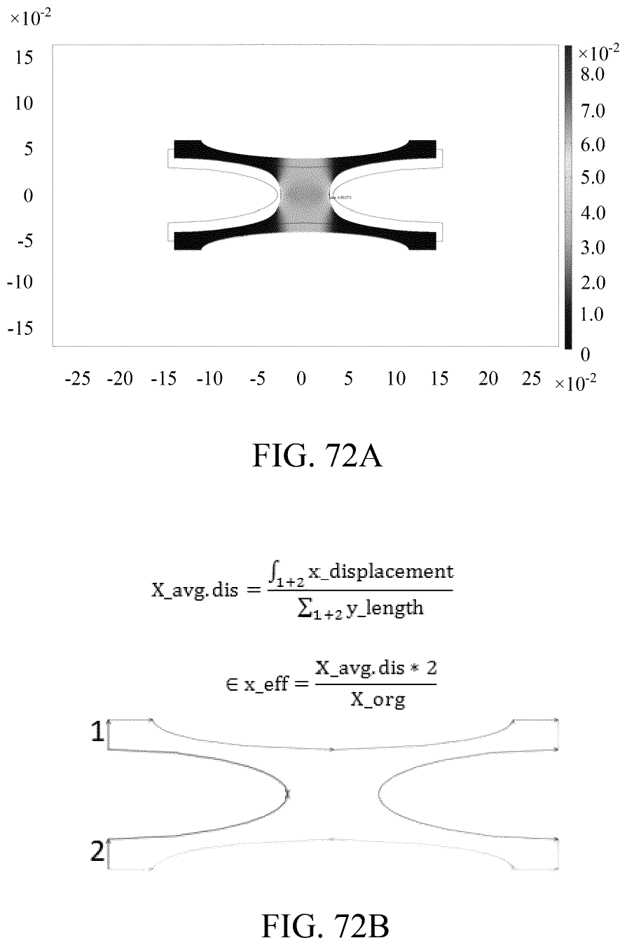

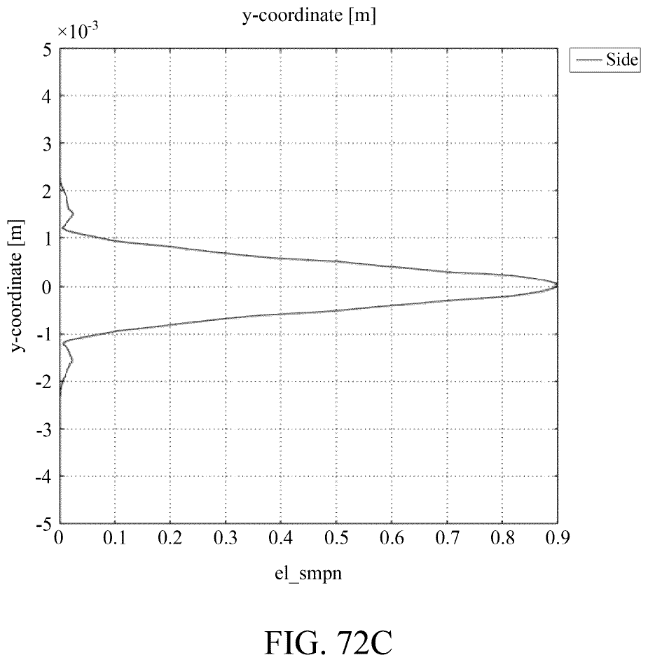

If modifying the area of the patch to the structure as shown in FIGS. 72A-72C. It can be seen that the drop is shown in Poisson's ratio and a non-uniform distribution of the strain across the area is also shown. That is, the drop strain is shown in most of the area in the patch. Max strain in structure: 88.5% and Effective Poisson's ratio=−ex/ey=0.2161. The key is in having a central rigid anchor (turquois color region) point to take up all the load while allowing the four long tapering elastic arms to stretch so that the overall structure can be expand more easily as shown in FIGS. 72A-72C. If a vertical load is applied, these arms are allowed to stretched and move easily in both vertical and horizontal directions.

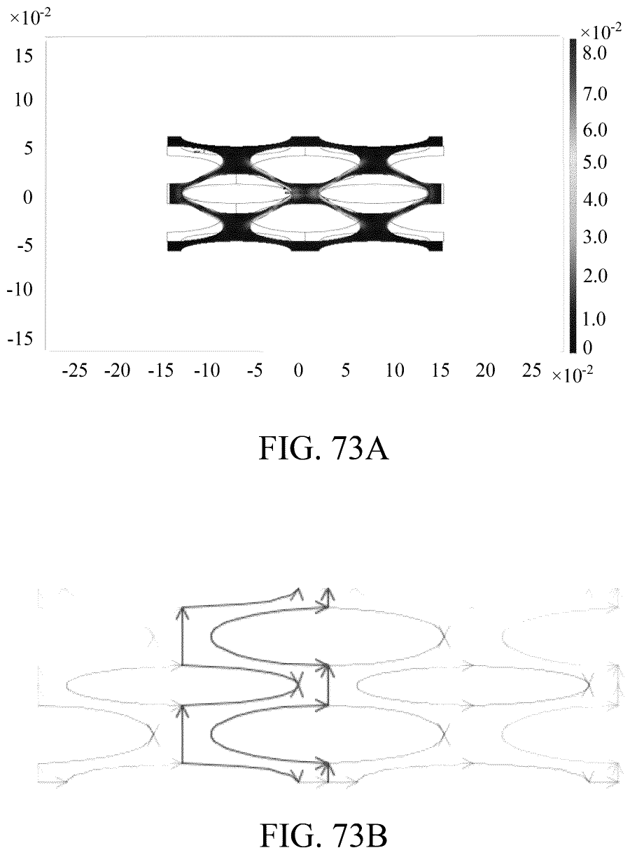

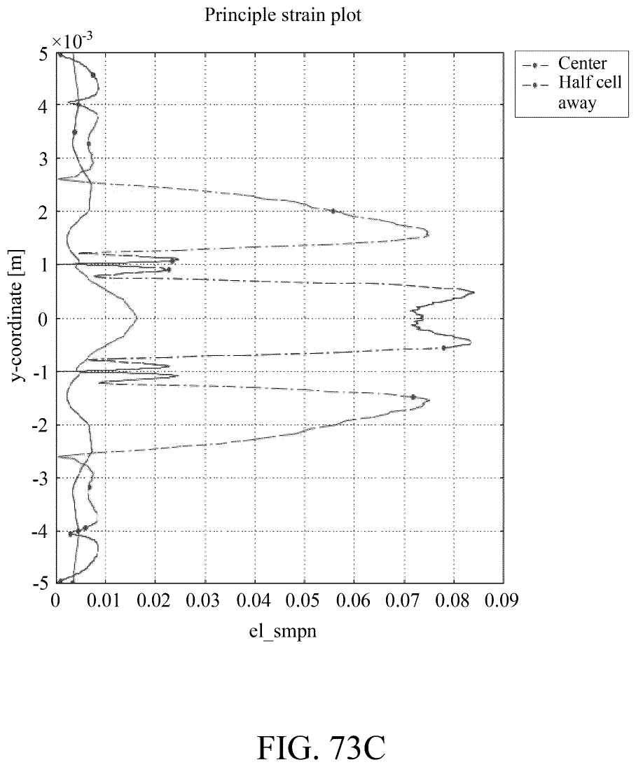

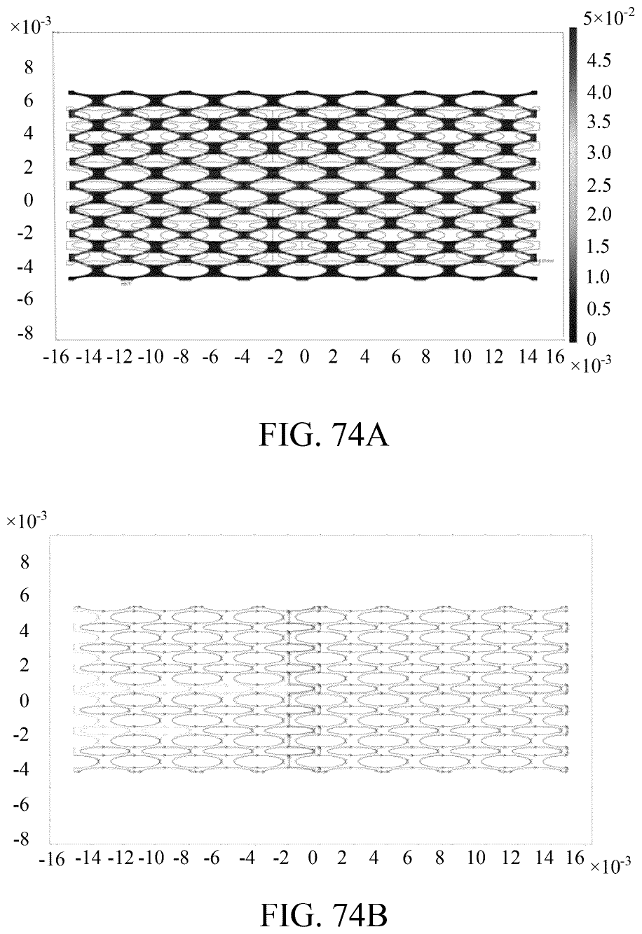

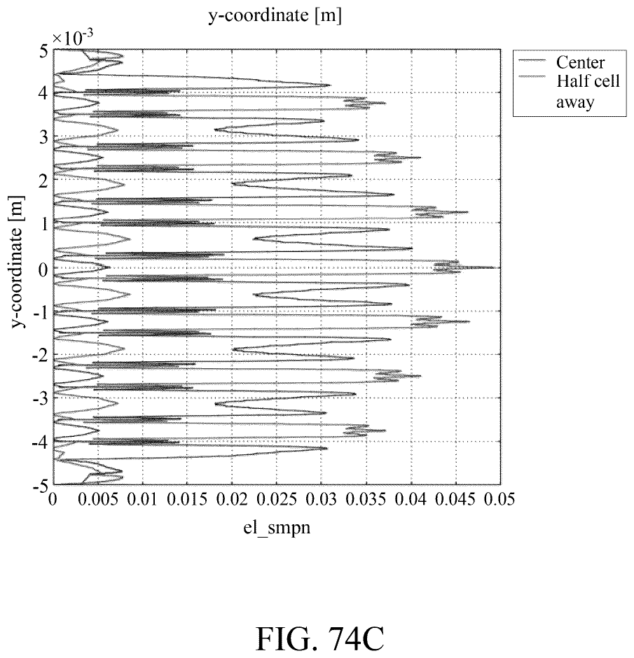

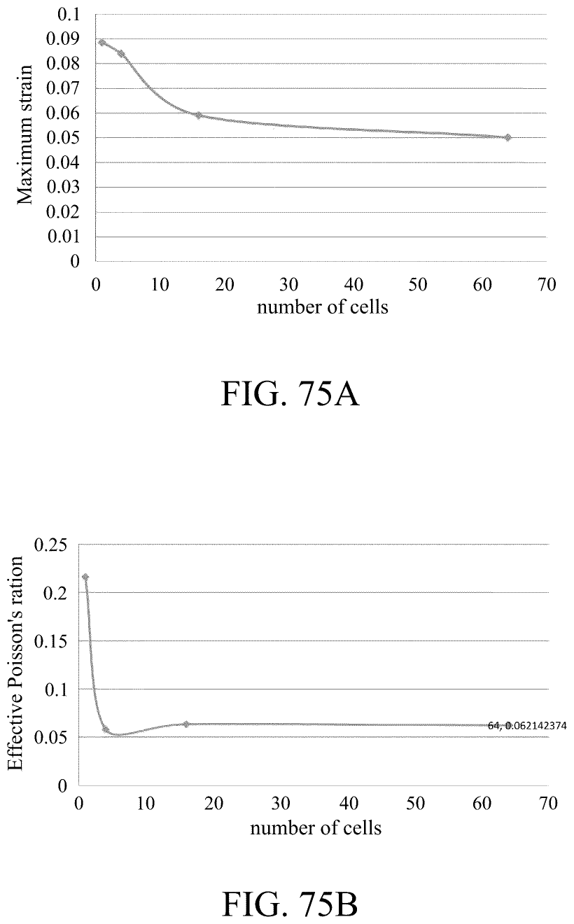

By further increasing the number of holes in the patch as shown in FIGS. 73A-73C and 74A-74C, it can be seen that localized strain is further reduce and Poisson's ratio starts to settle at a very low value around 0.06. With 64 cells design, it can be seen that the maximum strain is around 5%, far cry from the original one cell 88% and 20% from uniform structure. The Poisson's ratio is also reduced to 0.06 (see FIGS. 75A and 75B). This is great for patch sensor that intends to measure only one direction strain.

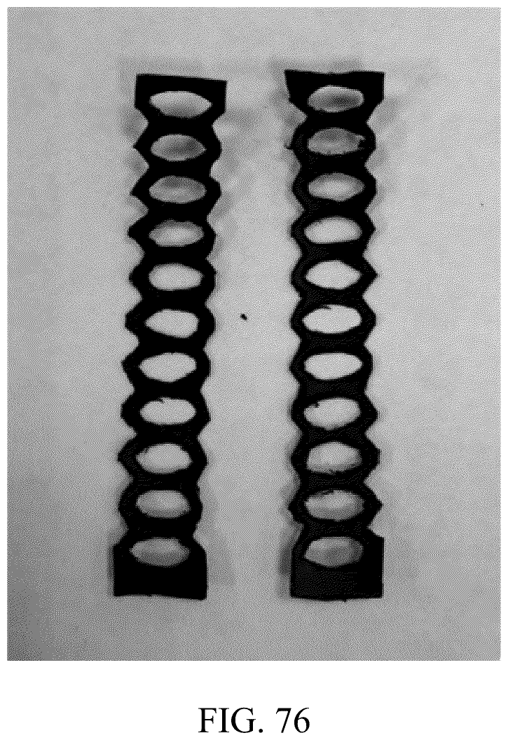

By increasing the aspect ratio between the width and height ratio of the hole in each cell, the strain enhancement is further increased. This idea is similar to archery bow design pattern where center of the bow is strong and the arms are taper so that it becomes more flexible and more easily to move. By combining several of these patterns and then roll it up, a tubular actuator is created, an accordion like expansion and contraction can be created, and very large overall strain on the structure can be generated. FIG. 76 shows a CNT/SMP composite using the strain enhancement design.

Electrode Deposition on Silicone Tube for Interconnect inside a Catheter System

To eliminate physical wiring of each individual actuator inside the soft robotic catheter, especially design shadow mask using rapid prototyping tool and typical sputtering metal deposition techniques are used to create these interconnects to simplify the power and sensor signal transport. The following shows an example of how this electrode can be designed and fabricated. However, many metal deposition in addition to sputter such as E-gun, electroplating, and earlier silver electrochemical plating as well as CNT and other polymer conductive polymers can also be used to create these electrodes either using direct deposition, spray on or di-pen process.

### Step 1: Measure the Tube Size and Design Mask

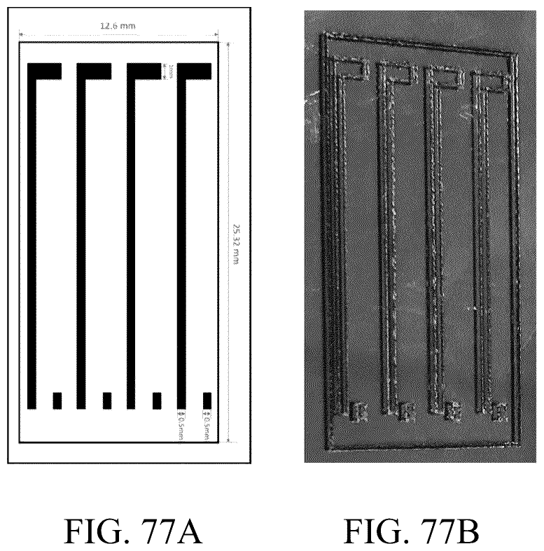

Before the mask is designed, it is needed to choose which size of the silicone tube is used. Since the silicone tube with the 4 mm diameter is used, circumference of the 4 mm diameter is 4×3.14159=12.6 mm. Accordingly, the width of the mask is 12.6 mm. As shown in FIG. 77A, four sets of electrodes are provided because four springs are attached on one section. In FIG. 77A, the L-shape electrode and the small rectangular electrode are one set. One is connected to positive and the other to negative/ground. FIG. 77B shows a CO₂ laser cut mask.

### Step 2: Making Masks (CO₂ Laser Cut the Pattern)

(a) Clean the wafer with IPA and rinse with the DI water.
(b) Paste a PI tape on wafer. It is needed to paste it carefully to prevent any bubbles.
(c) Import the pattern by using jpg or png file into the CO₂ laser software. The traveling speed of the laser may be set to 30 mm/s. Under this speed, heat generated by the laser would not burn the tape. (If less than 25 mm/s, tape burns) Besides, current of the laser has to be 22 mA. If using 21 mA, it won't cut nicely. However, if using 23 mA or higher, the PI tape starts to melt.
(d) Peel of the patterned mask (seen as a patterned sticker) and paste it surround the silicone tube. Once we paste it on the tube, only the pattern part expose to the air which will later be metal deposited.

### Step 3: Sputter Au on the Silicone Tube

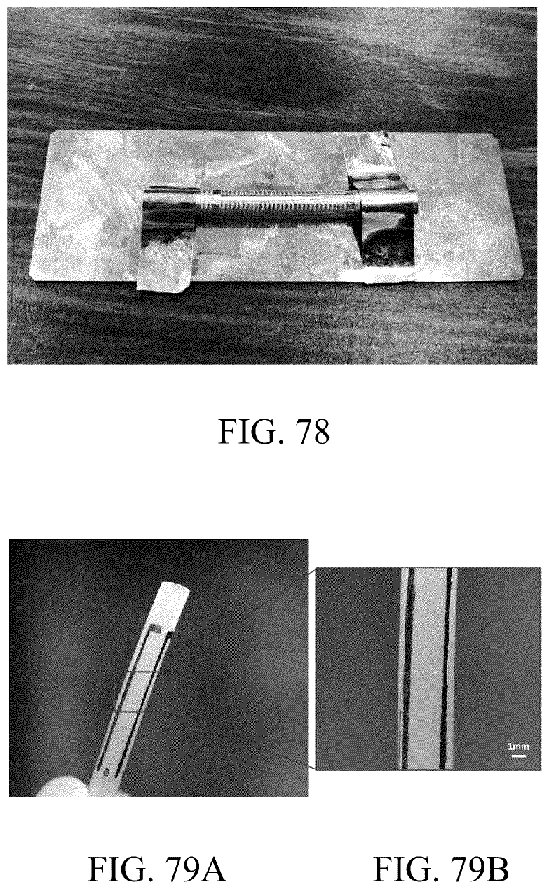

To create conductive electrode on the silicone tube, metal deposition technique we used is sputter. Since four sets of electrodes are provided, it is needed to do four times. Current is set to be 35 mA and the pressure is around 80 to 100 mTorr in the chamber. Target that we used is gold. For one side, it is deposited for 3 minutes: then go to the next side until four sides are all done. FIG. 78 shows a setup for depositing Au on a silicone tube. After sputtering, the mask can be peeled off.

### Step 4: Result

The electrodes result is shown in FIGS. 79A and 79B. After the deposition, a multimeter is used to check every line and every set of electrodes, everything is okay. L-shape electrode has high resistance around 250 to 350Ω.

### Sensor and Driving and Feedback Control

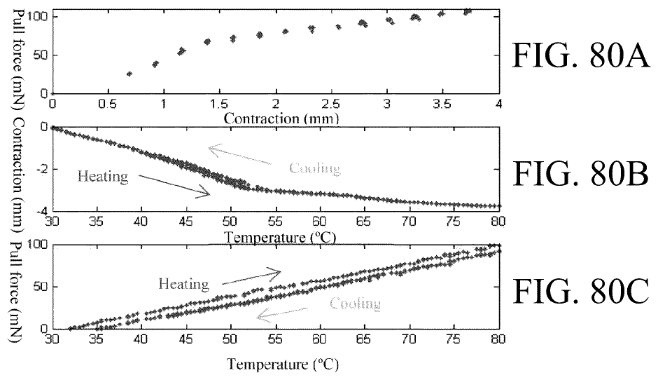

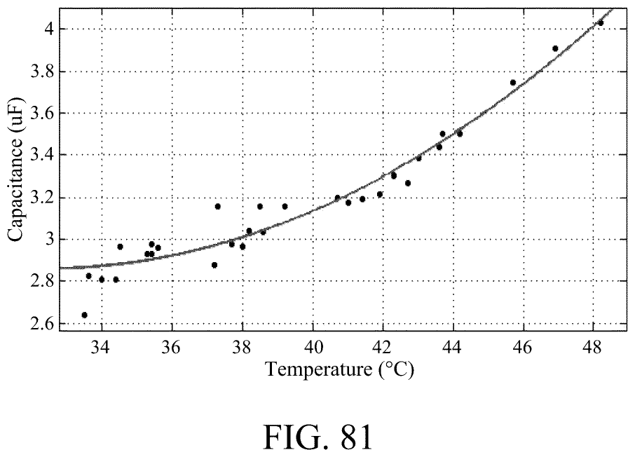

FIG. 80A shows a spring load curve at 25° C. FIG. 80B is a diagram showing a spring contraction as a function of temperature. FIG. 80C shows a pull force versus temperature diagram of a spring actuator. The actuator is also a capacitor, with varying capacitance due to bending or temperature changes in the spring (see FIG. 81). Because of this built-in sensing capability, the precise tip position and rotation can be found through careful calibration with known applied loads (temperature or bending) for each actuator array segment, similar to the curve from our previous spring sensor design. These sensors can also be used to create a close-loop feedback system to prevent the tip of the catheter from damaging the walls of the blood vessel. With this feedback, catheter insertion can be made safer and more reliable, reducing risk of injury to patients. In the present disclosure, 5 multilink active catheter arrays or tubular configurations are utilized, and the final design has additional sensors/actuators to improve mobility and resolution.

The capacitance detection circuit uses a LC circuit with an input range of 10⁶-10 uF and 0.1 pF sensitivity to prevent interference caused by contact and temperature fluctuation of the electrical contacts. If more channels are used, a multi-channel 24-Bit Capacitance-to-Digital Converter (AD7746, Analog Devices) will replace the analog LC circuit to increase sampling speed and sensitivity.

### Integrated Sensors, Actuators and Control Circuitry

The shape-shifting actuator system of the present disclosure requires an effective power control system. A compact, high-current power supply with an energy regulator is developed to provide adjustable input amplitude and pulse widths. Switching is handled by inverting Schmitt triggers for tunable pulse frequency and duty cycle to regulate output current from 1 mA to 1 A. The charge and discharge time depend on the shape-shifting materials.

The power supply design is flexible enough for different operating conditions and different materials, including arrays of large modules. A 3.7 V 9900 mAh Lithium ion battery will power the system. Operating at full load, the converter drains approximately 400 mA. At the current pulse rate and duty cycle, the battery should last at least a day. The first prototype will use 20 active modules, so the battery should last ˜25 hours of consecutive use. The system will hibernate during inactivity, prolonging battery life. The power consumption can be considerably lowered to allow for continuous use without a daily recharge.

Future control of the sensor/actuator modules will include a field programmable gate array (FPGA), which offers quick prototyping and parallel algorithm execution for simultaneous control of large numbers of the sensor/actuator modules. With the FPGA, the positions of the modules can be maintained by a voltage detection circuit that will compensate for voltage drops below the operational range.

One concern with the shape-shifting polymer is that it operates slightly above body temperature and electromagnetic radiation. This issue can be solved in the circuit designs shielding for heat and EM radiations. The actuator requires only a few milliamps such that the power supply circuit will be limited only a few milliwatts of power and millijoules of heat, posing no hazard to human operators or patients. The shape-shifting polymers are non-abrasive, non-toxic, and non-polluting (meeting health and safety regulations).

The descriptions of the various embodiments of the present disclosure have been presented for purposes of illustration but are not intended to be exhaustive or limited to the embodiments disclosed. Many modifications and variations will be apparent to those of ordinary skill in the art without departing from the scope and spirit of the described embodiments. The terminology used herein is chosen to best explain the principles of the embodiments, the practical application or technical improvement over technologies found in the marketplace, or to enable others of ordinary skill in the art to understand the embodiments disclosed herein.

What is claimed is:

1. A multi-degree-of-freedom steerable catheter soft robotic system, comprising:
a steerable catheter;
a control circuit connected to the steerable catheter through electrical connections and selectively applying power to control the steerable catheter; and
a power supply unit connected to the control circuit.

2. The multi-degree-of-freedom steerable catheter soft robotic system of claim 1, further comprising a driving circuit for driving the steerable catheter.

3. The multi-degree-of-freedom steerable catheter soft robotic system of claim 1, further comprising a shielding disposed around the steerable catheter and shielding for heat and electromagnetic (EM) radiations.

4. The multi-degree-of-freedom steerable catheter soft robotic system of claim 1, wherein the steerable catheter comprises a plurality of tubular segments, and each of the tubular segments comprises a plurality of self-sensing shape-shifting spring coil actuators.

5. The multi-degree-of-freedom steerable catheter soft robotic system of claim 4, wherein the plurality of self-sensing shape-shifting spring coil actuators are evenly spaced inside each of the tubular segments.

6. The multi-degree-of-freedom steerable catheter soft robotic system of claim 4, wherein a seed layer is deposited on a surface of each of the plurality of self-sensing shape-shifting spring coil actuators by an electroless silver plating process.

7. The multi-degree-of-freedom steerable catheter soft robotic system of claim 6, wherein a silver layer is deposited on a surface of the seed layer by the electroless silver plating process.

8. The multi-degree-of-freedom steerable catheter soft robotic system of claim 1, wherein the steerable catheter comprises a self-sensing shape-shifting memory polymer (SMP) actuator without conductive coating agents or with conductive coating agents by a silver chemical plating process or a carbon nanotube (CNT) composite process.

9. The multi-degree-of-freedom steerable catheter soft robotic system of claim 8, wherein the SMP actuator is made electrically and thermally conductive by the silver chemical plating process such that the SMP actuator is a self-sensing silver plated SMP actuator.

10. The multi-degree-of-freedom steerable catheter soft robotic system of claim 8, wherein the SMP actuator is made electrically and thermally conductive by the carbon nanotube (CNT) composite process such that the SMP actuator is a self-sensing CNT-based SMP actuator.

11. The multi-degree-of-freedom steerable catheter soft robotic system of claim 8, wherein the SMP actuator is bent upward or downward to show flexibility by utilizing a multi-phase shape-shifting memory material.

12. The multi-degree-of-freedom steerable catheter soft robotic system of claim 11, wherein the multi-phase shape-shifting memory material is a self-sensing and reversible LC elastomer.

13. The multi-degree-of-freedom steerable catheter soft robotic system of claim 11, wherein the multi-phase shape-shifting memory material is a self-sensing and reversible bi-layer composite sheet, and the self-sensing and reversible bi-layer composite sheet comprises a self-sensing and reversible CNT-based SMP together with a polyurethane (PU), polyimide (PI) or polyester (PET) film.

14. The multi-degree-of-freedom steerable catheter soft robotic system of claim 8, wherein the SMP actuator is bent upward and downward by a pneumatic process.

15. The multi-degree-of-freedom steerable catheter soft robotic system of claim 8, wherein a Negative Poisson's Ratio (NPR) structure is used in the SMP actuator such that auxetics and strains of the SMP actuator are enhanced.

16. A method of manufacturing a steerable catheter of a multi-degree-of-freedom steerable catheter soft robotic system, the method comprising:
forming a plurality of tubular segments;
forming a plurality of self-sensing shape-shifting spring coil actuators inside each of the plurality of tubular segments;
depositing a seed layer on a surface of each of the plurality of self-sensing shape-shifting spring coil actuators by an electroless silver plating process; and
depositing a silver layer on a surface of the seed layer by the electroless silver plating process.

17. The method of claim 16, wherein the electroless silver plating process comprises:
immersing a spring coil into sodium hydroxide (NaOH) in a beaker;
placing the spring coil into an ultrasonic washing machine for few minutes;
performing an Iodine pretreatment on the spring coil;
immersing the spring coil an Au etchant under room temperature;
placing the spring coil into Sodium Borohydride (NaBH4); and
rinsing the spring coil to remove any chemicals and residue out of a surface thereof such that the seed layer is formed on a surface of the spring coil.

18. The method of claim 17, wherein the electroless silver plating process further comprises forming the silver layer on the surface of the seed layer such that the spring coil becomes the self-sensing shape-shifting spring coil actuator.

19. A method of manufacturing a steerable catheter of a multi-degree-of-freedom steerable catheter soft robotic system, the method comprising:
forming and coating a self-sensing shape-shifting memory polymer (SMP) actuator without conductive coating agents or with conductive coating agents by a silver chemical plating process or a carbon nanotube (CNT) composite process.

20. The method of claim 19, wherein the silver chemical plating process comprises:
providing a solution;
pouring the solution into a mold;
heating the mold by a curing process;
forming a sheet after the solution is fully cured;
removing the sheet from the mold; and
cutting a window array pattern on the sheet,
wherein the solution is one of a polyurethane (PU)-based shape memory polymer solution, a polyimide (PI)-based shape memory polymer solution and a polyester (PET)-based shape memory polymer solution.

21. The method of claim 20, wherein the silver chemical plating process further comprises:
depositing a conductive layer on a surface of the sheet; and
rolling the sheet into a tube.

22. The method of claim 20, wherein the window array pattern is a rectangular window array pattern, a re-entrant honeycomb window array pattern, a chiral honeycomb window array pattern, a rotating rectangle window array pattern or a combination thereof.

23. The method of claim 19, wherein the CNT composite process comprises:
providing a solution;
pouring the solution into a mold;
heating the mold by a curing process;
forming a sheet after the solution is fully cured;
removing the sheet from the mold; and
cutting a window array pattern on the sheet,
wherein the solution is provided by mixing liquid phase SMP, dimethylformamide (DMF) and CNT powders together with a weight ratio.

24. The method of claim 23, wherein the CNT composite process further comprises:
depositing a conductive layer on a surface of the sheet; and
rolling the sheet into a tube.

25. The method of claim 23, wherein the window array pattern is a rectangular window array pattern, a re-entrant honeycomb window array pattern, a chiral honeycomb window array pattern, a rotating rectangle window array pattern or a combination thereof.

26. The method of claim 23, wherein the sheet is one of a self-sensing and reversible LC elastomer and a self-sensing and reversible bi-layer composite sheet.

27. The method of claim 26, wherein the self-sensing and reversible bi-layer composite sheet comprises a self-sensing and reversible CNT-based SMP together with a polyurethane (PU), polyimide (PI) or polyester (PET) film.

28. The method of claim 27, wherein a linkage structure is provided by a combination of a mechanism structure and the self-sensing and reversible CNT-based SMP.

29. A method of operating a steerable catheter of a multi-degree-of-freedom steerable catheter soft robotic system, the method comprising:
providing a silicone tube and a steerable catheter;
connecting a portion of the silicon tube to a nitrogen gas tank with a pressure control valve and connecting another portion of the silicone tube to into steerable catheter;
releasing nitrogen gas into the silicon tube to enhance stiffness of the silicon tube; and
gradually increasing an air pressure to straighten the steerable catheter.

30. The method of claim 28, wherein when no gas is released into the silicon tube, the silicon tube remains soft such that the steerable catheter is bent upward or downward.

31. The method of claim 28, wherein the steerable catheter is a self-sensing and reversible carbon nanotube (CNT)-based SMP actuator.
